SEQUENCE LISTING (50 sequences)

SEQ ID NO: 1 - DNA, 21 bp, Artificial Sequence
  forward primer for qPCR of viral immediate-early protein ICP0 DNA
ataagttagccctggccccga

SEQ ID NO: 2 - DNA, 16 bp, Artificial Sequence
  reverse primer for qPCR of viral immediate-early protein ICP0 DNA
gctgcgtctcgctccg

SEQ ID NO: 3 - DNA, 20 bp, Artificial Sequence
  forward primer for qPCR of mouse Tbx15 DNA
tcccccttctcttgtgtcag

SEQ ID NO: 4 - DNA, 20 bp, Artificial Sequence
  reverse primer for qPCR of mouse Tbx15 DNA
cggaagcaagtctcagatcc

SEQ ID NO: 5 - DNA, 21 bp, Artificial Sequence
  forward primer for qRT-PCR of mouse gene Ifit1
agcaaccatgggagagaatgc

SEQ ID NO: 6 - DNA, 20 bp, Artificial Sequence
  reverse primer for qRT-PCR of mouse gene Ifit1
cctttcaggtgcctcacgta

SEQ ID NO: 7 - DNA, 20 bp, Artificial Sequence
  forward primer for qRT-PCR of mouse gene Irf7
agcttggatctactgtgcgc

SEQ ID NO: 8 - DNA, 20 bp, Artificial Sequence
  reverse primer for qRT-PCR of mouse gene Irf7
gggttcctcgtaaacacggt

SEQ ID NO: 9 - DNA, 20 bp, Artificial Sequence
  forward primer for qRT-PCR of mouse gene Oasl2
tgcctgggagagaatcgaag

SEQ ID NO: 10 - DNA, 20 bp, Artificial Sequence
  reverse primer for qRT-PCR of mouse gene Oasl2
agcctcccttcaccacctta

SEQ ID NO: 11 - DNA, 20 bp, Artificial Sequence
  forward primer for qRT-PCR of mouse gene Rps29
ggtcaccagcagctctactg

SEQ ID NO: 12 - DNA, 25 bp, Artificial Sequence
  reverse primer for qRT-PCR of mouse gene Rps29
gtccaacttaatgaagcctatgtcc

SEQ ID NO: 13 - DNA, 19 bp, Artificial Sequence
  forward primer for qRT-PCR of mouse gene Stat1
ggatcgcttgcccaactct

SEQ ID NO: 14 - DNA, 21 bp, Artificial Sequence
  reverse primer for qRT-PCR of mouse gene Stat1
gcagagctgaaacgacctaga

SEQ ID NO: 15 - DNA, 20 bp, Artificial Sequence
  forward primer for qRT-PCR of mouse gene Rsad2
ggaggtggtgcagggattac

SEQ ID NO: 16 - DNA, 20 bp, Artificial Sequence
  reverse primer for qRT-PCR of mouse gene Rsad2
ggaaaaccttccagcgcaca

SEQ ID NO: 17 - DNA, 20 bp, Artificial Sequence
  forward primer for qRT-PCR of mouse gene Stat2
cttttgcaagcgagagagcc

SEQ ID NO: 18 - DNA, 20 bp, Artificial Sequence
  reverse primer for qRT-PCR of mouse gene Stat2
tgaagcgcagtaggaaggtg

SEQ ID NO: 19 - DNA, 23 bp, Artificial Sequence
  forward primer for qRT-PCR of mouse gene Ifn-alpha
tctctccacactttgtctcacac

SEQ ID NO: 20 - DNA, 22 bp, Artificial Sequence
  reverse primer for qRT-PCR of mouse gene Ifn-alpha
acagtccagagagccatcaacc

SEQ ID NO: 21 - DNA, 20 bp, Artificial Sequence
  forward primer for qRT-PCR of mouse gene Ifn-beta
aagatctctgctcggaccac

SEQ ID NO: 22 - DNA, 20 bp, Artificial Sequence
  reverse primer for qRT-PCR of mouse gene Ifn-beta
tgggagatgtcctcaactgc

SEQ ID NO: 23 - DNA, 21 bp, Artificial Sequence
  forward primer for qRT-PCR of mouse gene Il28a
gcagacctgtacacagcttca

SEQ ID NO: 24 - DNA, 20 bp, Artificial Sequence
  reverse primer for qRT-PCR of mouse gene Il28a
caggttggaggtgacagagg

SEQ ID NO: 25 - DNA, 20 bp, Artificial Sequence
  forward primer for qRT-PCR of human gene IFIT1
agcttacaccattggctgct

SEQ ID NO: 26 - DNA, 23 bp, Artificial Sequence
  reverse primer for qRT-PCR of human gene IFIT1
ccatttgtactcatggttgctgt

SEQ ID NO: 27 - DNA, 18 bp, Artificial Sequence
  forward primer for qRT-PCR of human gene IRF7
agctgtgctggcgagaag

SEQ ID NO: 28 - DNA, 20 bp, Artificial Sequence
  reverse primer for qRT-PCR of human gene IRF7
ctctccaggagccttggttg

SEQ ID NO: 29 - DNA, 20 bp, Artificial Sequence
  forward primer for qRT-PCR of human gene RPLP0
ccacattgtctgctcccaca

SEQ ID NO: 30 - DNA, 20 bp, Artificial Sequence
  reverse primer for qRT-PCR of human gene RPLP0
gaagacagggcgacctggaa

SEQ ID NO: 31 - DNA, 21 bp, Artificial Sequence
  forward primer for qRT-PCR of human gene RSAD2
gctgctagctaccaagaggag

SEQ ID NO: 32 - DNA, 21 bp, Artificial Sequence
  reverse primer for qRT-PCR of human gene RSAD2
atcttctccataccagcttcc

SEQ ID NO: 33 - DNA, 21 bp, Artificial Sequence
  forward primer for qRT-PCR of human gene STAT1
aaaggaagcaccagagccaat

SEQ ID NO: 34 - DNA, 20 bp, Artificial Sequence
  reverse primer for qRT-PCR of human gene STAT1
tccgagacacctcgtcaaac

SEQ ID NO: 35 - DNA, 20 bp, Artificial Sequence
  forward primer for qRT-PCR of human gene STAT2
ggatcctacccagttggctg

SEQ ID NO: 36 - DNA, 20 bp, Artificial Sequence
  reverse primer for qRT-PCR of human gene STAT2
gagggtgtcttccctttggc

SEQ ID NO: 37 - DNA, 20 bp, Artificial Sequence
  forward primer for qRT-PCR of human gene OAS2
gggctatttccagacaacgc

SEQ ID NO: 38 - DNA, 23 bp, Artificial Sequence
  reverse primer for qRT-PCR of human gene OAS2
gaaaaccaggcctgtgatcttgg

SEQ ID NO: 39 - DNA, 20 bp, Artificial Sequence
  forward primer for qRT-PCR of human gene OAS1
ttcctccctgccattcatcc

SEQ ID NO: 40 - DNA, 21 bp, Artificial Sequence
  reverse primer for qRT-PCR of human gene OAS1
tccagaaaccctcgattgtga

SEQ ID NO: 41 - DNA, 20 bp, Artificial Sequence
  forward primer for qRT-PCR of human gene MxA
acctacagctggctcctgaa

SEQ ID NO: 42 - DNA, 20 bp, Artificial Sequence
  reverse primer for qRT-PCR of human gene MxA
gcactcaagtcgtcagtcca

SEQ ID NO: 43 - DNA, 21 bp, Artificial Sequence
  forward primer for qRT-PCR of human gene RPL27
aaagctgtcatcgtgaagaac

SEQ ID NO: 44 - DNA, 21 bp, Artificial Sequence
  reverse primer for qRT-PCR of human gene RPL27
gctgctactttgcgggggtag

SEQ ID NO: 45 - DNA, 21 bp, Artificial Sequence
  forward primer for amplification of the genomic DNA for beta-actin
tactcctgcttgctgatccac

SEQ ID NO: 46 - DNA, 20 bp, Artificial Sequence
  reverse primer for amplification of the genomic DNA for beta-actin
tgtgtggggagctgtcacat

SEQ ID NO: 47 - DNA, 20 bp, Artificial Sequence
  forward primer for amplification of the DNA for viral glycoprotein B
cgcatcaagaccacctcctc

SEQ ID NO: 48 - DNA, 15 bp, Artificial Sequence
  reverse primer for amplification of the DNA for viral glycoprotein B
gctcgcaccacgcga

SEQ ID NO: 49 - DNA, 20 bp, Artificial Sequence
  forward primer for qRT-PCR of ZIKV
agatcccggctgaaacactg

SEQ ID NO: 50 - DNA, 20 bp, Artificial Sequence
  reverse primer for qRT-PCR of ZIKV
ttgcaaggtccatctgtccc

(19) United States
(12) Patent Application Publication
(10) Pub. No.: US 2021/0322404 A1
(43) Pub. Date: Oct. 21, 2021

(54) FGFR REGULATION FOR THE TREATMENT OF VIRAL INFECTIONS

(71) Applicant: ETH ZURICH, Zurich (CH)

(72) Inventors: SABINE WERNER, Zurich (CH); Michael MEYER, Zurich (CH); Luigi MADDALUNO, Zurich (CH)

(21) Appl. No.: 16/341,770

(22) PCT Filed: Oct. 12, 2017
(86) PCT No.: PCT/EP2017/076102

§ 371 Date: Apr. 12, 2019

(30) Foreign Application Priority Data
Oct. 13, 2016 (EP) 16193748.7

(51) Int. Cl. A61K 31/496; A61K 31/5025; A61K 31/47; A61K 31/53; A61K 31/501; A61K 31/498; A61K 31/404; A61K 38/17; A61K 47/68; A61K 39/395; A61K 31/4439; A61K 31/4184; A61K 31/519; A61P 31/22; A61P 31/14

(52) U.S. Cl. CPC A61K 31/496; A61P 31/14; A61K 31/47; A61K 31/53; A61K 31/501; A61K 31/498; A61K 31/404; A61K 38/179; A61K 47/6811; A61K 39/3955; A61K 31/4439; A61K 31/4184; A61K 31/519; A61P 31/22; A61K 31/5025

(57) ABSTRACT

The present invention is directed to a compound for inhibiting (i) FGF-R kinase activity or (ii) a component of the FGFR kinase signaling pathway for use in the therapeutic or prophylactic treatment of viral infections as well as corresponding pharmaceutical compositions for use in the same treatment and related methods of treatment.

The present invention is directed to a compound for inhibiting (i) FGFR kinase activity or (ii) a component of the FGFR kinase signaling pathway for use in the therapeutic or prophylactic treatment of viral infections as well as corresponding pharmaceutical compositions for use in the same treatment and related methods of treatment.

### BACKGROUND

Viral infections are widespread and a major health risk for humans and animals worldwide because they are generally difficult to treat. In most cases, the treatment of viral infections consists of reducing the symptoms by antipyretic and analgesic drugs, by avoiding exposure and reducing contamination, e.g. by disinfection. Also, there are vaccines for some viruses, which, of course, are only effective if administered prior to an infection.

For a limited number of viruses, virostatic medicaments are employed, which inhibit the production of new viruses. These virostatic agents, for example, interfere with the viral life cycle before cell entry, during viral synthesis or assembly or during the release phase from the host cell. A general draw-back of current virostatic medicaments is their virus-specific nature, their susceptibility to viral variation and/or their toxicity.

For example, the very common Herpes simplex virus (HSV) 1 affects the skin and the genital tract. Current treatment options involve the inhibition of the thymidine kinase of HSV by the administration of nucleoside analogs or the administration of Helicase-primase inhibitors, which are associated with certain toxicity, major side effects and mutagenic potential. Also, the virostatic treatment of many HSV infections is presently limited to the systemic administration of the medicaments and there are limited efficient topical or local treatment options.

Van et al. (Van et al., Gut 65, 1015-1023, 2016) investigated the modulation of hepatitis C virus (HCV) reinfection after orthotopic liver transplantation (OLT) by fibroblast growth factor-2 (FGF2) and other non-interferon mediators in the context of liver cirrhosis or hepatocellular carcinoma (HCC). Van et al. speculate that sera from post-OLT patients contained one or more factor(s) that enhance HCV infectivity (page 1017, right column, 2^{nd} paragraph). However, when characterizing the sera, Van et al. found that the change in individual mediators was highly heterogeneous between patients and the change did not reach statistical significance for any of the mediators and they could not find any clear pattern of mediators seen in those individuals whose post-OLT sera enhanced HCV in vitro infectivity (page 1017, right column, 3^{rd} paragraph). By randomly screening different serum components, Van et al. found that FGF2 enhanced viral replication and new particle production, but not infection with the virus.

Van et al. allege that FGF2 is dependent on signaling through FGF receptor (FGFR)3, which is not present in all tissues. In this regard, Van et al. find that another member of the FGF family, FGF1, does not enhance HCV replication (see FIG. 4 of Van et al.) in hepatic cells. This is surprising, since FGF1 binds to all FGFRs (Zhang et al., J. Biol. Chem. 281, 15694-15700, 2006), and the lack of activity of FGF1 in this experiment may be related to the overall lower biological activity of FGF1 compared to FGF2 or to the use of an insufficiently potent preparation. Importantly, hepatocytes bear a distinct FGF receptor expression pattern and it cannot be predicted whether the signaling in hepatocytes with regard to FGF and its receptor target and anti-HCV effects could be transferred to any other tissues or to any other viruses. In addition to FGFR3, hepatocytes also express high levels of FGFR4 and lower levels of FGFR2 and FGFR1, and it remains to be determined if inhibitors of other FGFRs have a similar effect. In particular, Van et al. are silent on the mechanism that could be involved in FGF2's influence on HCV in liver cells. It is unknown which (if any) cellular proteins downstream of the FGF receptor are involved and responsible for the observations made in Van et al.

It is the objective of the present invention to provide new means for the treatment of viral infections.

In a first aspect, the above objective is solved by a compound for inhibiting (i) FGFR kinase activity and/or (ii) a component of the FGFR kinase signaling pathway for use in the treatment of viral infections.

It was surprisingly found that FGF signaling dramatically increases viral replication by blocking the transcription of classical interferon regulated genes. Furthermore, it was found that loss of FGFR kinase activity or its downstream targets significantly reduce viral replication also in the presence of e.g. FGF7, the classical ligand of FGFR2b. It was also found that the antiviral effect of FGFR downregulation/kinase inhibition is due to a strong induction of various interferon response genes, which encode proteins that inhibit different stages of the viral life cycle. Therefore, FGFR inhibition is suitable for use in the treatment of viral diseases. Without wishing to be bound by theory, it is believed that the interferon response efficiently inhibits infection by and replication of many different types of viruses. Therefore, most of the compounds for use in the present invention are generally more efficient and more widely applicable compared to currently used virostatic medicaments. Another advantage of FGFR inhibitors, e.g. kinase inhibitors, ligand traps or neutralizing antibodies, is that they are in or have already gone through clinical trials for cancer prevention and have been found to be well-tolerated even upon long-term applications. Therefore, they have less side effects compared to the currently used virostatic medicaments.

Also, the compound for inhibiting (i) FGFR kinase activity and/or (ii) a component of the FGFR kinase signaling pathway can be for use in the treatment of most, if not all viral infections of mammals, in particular humans, independent of the type of virus and the target tissue(s).

By way of example, it was found that various interferon target genes are under direct control of FGFs and these FGFs were identified as efficient inhibitors of interferon signaling through an FGFR signaling pathway involving Rac1 and p38 (see further below). Without wishing to be bound by theory, these results demonstrate the relevance of FGFR kinase pathways, for example the FGFR kinase-Rac1 pathway, for viral replication and, thus, identify FGFs/FGF receptors as novel targets for antiviral therapies.

As used herein, the term “treatment” refers to both, prophylactic and/or therapeutic treatment unless the type of treatment is specified as prophylactic or therapeutic.

The term “inhibiting” in the context of inhibiting FGFR kinase activity and/or inhibiting a component of the FGFR kinase signaling pathway is understood to include at least partial reduction, preferably complete loss of the inherent biological function of said FGFR receptor or FGFR pathway component, including kinases and other proteins. In the context of the present invention, the reduction in biological function/activity of the FGFR kinase and/or FGFR pathway component must be to such an extent that a virus infection is significantly affected by the said reduction.

In a preferred embodiment, the composition for use according to the present invention is a composition, wherein the compound (i) for inhibiting FGFR kinase activity is selected from the group consisting of
(a) FGFR kinase inhibitors: AZD4547, Ponatinib, Dovitinib, Nintedanib, Lenvatinib, Lucitanib, Brivanib, ENMD-2076, BGJ398, FGF401, Lucitanib, PD173074, SU5402, SSR128129E, ARQ 087, LY2874455, Debio 1347, TAS-120, Erdafitinib, Nintedanib and Orantinib; FGFR ligand traps: FP1039; and FGFR neutralizing antibodies: IMC-A1, PRO-001, R3Mab, FPA144, and MGFR1877S;
(b) preferably AZD4547, BGJ398, LY2874455, Debio 1347, TAS-120, Erdafitinib, FPA144 and FP1039;
(c) more preferably AZD4547 and BGJ398; and
(d) a physiologically acceptable salt of (a), (b) and (c).

The present invention includes physiologically acceptable salts or solvates of the compounds for use in the present invention. A “physiologically acceptable salt” refers to any physiologically acceptable salt or solvate which, upon administration to a patient, is capable of providing (directly or indirectly) (a) a compound for use according to the present invention, or (b) a pharmacologically active metabolite or pharmacologically active residue thereof. A pharmacologically active metabolite shall be understood to mean any compound being metabolized enzymatically or chemically to result in a compound for use in the present invention.

Physiologically acceptable salts include those derived from physiologically acceptable inorganic and organic acids and bases. Examples of suitable acids include, but are not limited to hydrochloric, hydrobromic, sulphuric, nitric, perchloric, fumaric, maleic, phosphoric, glycolic, lactic, salicylic, succinic, toluene-p-sulfuric, tartaric, acetic, citric, methanesulfonic, formic, benzoic, malonic, naphthalene-2-sulfuric and benzenesulfonic acids. Other acids, such as oxalic acid, while not themselves physiologically acceptable, may be employed in the preparation of salts useful as intermediates in obtaining the compounds and their physiologically acceptable acid addition salts. Salts derived from appropriate bases include alkali metals, preferably lithium, sodium, potassium or cesium; alkaline earth metals, preferably magnesium or calcium; ammonium, N—(C₁-C₄ alkyl)₄⁺, manganese, iron, nickel, copper, zinc or aluminium salts.

In a further preferred embodiment, the composition for use according to the present invention is a composition, wherein the compound for (ii) inhibiting a component of the FGFR kinase signaling pathway is selected from the group consisting of
(a) RAC1-inhibiting compounds, preferably selected from the group consisting of NSC23766, EHop-016, Azathioprine, EHT 1864;
(b) p38 MAPK-inhibiting compounds, preferably selected from the group consisting of SB203580, VX-702, VX-745, Pamapimod, Iosmapimod, Dilmapimod, Doramapimod, BMS582949, ARRY-797, PH797804, SC10-469, SD-0006, AMG-548, LY2228820, SB239063, Skepinone L, SB202190 and TAK715; and
(c) a physiologically acceptable salt of (a) and (b).

In a further preferred embodiment, the present invention relates to a composition for use in the treatment of viral infections, preferably HSV1, HSV2, Lymphocytic Choriomeningitis virus (LCMV) or Zika Virus infections, wherein the compound for (ii) inhibiting a component of the FGFR kinase signaling pathway is selected from the group consisting of
(a) RAC1-inhibiting compounds, preferably selected from the group consisting of NSC23766, EHop-016, Azathioprine, EHT 1864, more preferably NSC23766; and
(b) a physiologically acceptable salt of (a).

In another preferred embodiment, the present invention relates to a composition comprising (A) a compound for inhibiting (i) FGFR kinase activity and/or (ii) a component of the FGFR kinase signaling pathway and (B) acyclovir (CAS 59277-89-3) for use in treating viral, preferably HSV1 or HSV2 infections.

For ease of reading, the following table provides for alternative and IUPAC names as well as CAS numbers for the specifically cited compounds for use in the present invention. Of course, antibodies do not have CAS numbers and often do not feature alternative names.

| | Alternative | | | CAS |
|---|---|---|---|---|
| Compound | name | Company | IUPAC name | number |
| AZD4547 | | AstraZeneca UK | N-[5-[2-(3,5-dimethoxyphenyl)ethyl]- | 1035270- |
| | | Ltd. | 1H-pyrazol-3-yl]-4-[(3R,5S)-3,5- | 39-3 |
| | | | dimethylpiperazin-1-yl]benzamide | |
| Ponatinib | AP24534 | Ariad | 3-(2-imidazo[1,2-b]pyridazin-3- | 943319- |
| | | Pharmaceuticals, | ylethynyl)-4-methyl-N-[4-[(4- | 70-8 |
| | | Inc., USA | methylpiperazin-1-yl)methyl]-3- | |
| | | | (trifluoromethyl)phenyl]benzamide | |
| Dovitinib | TKI258 | Novartis AG, | (3Z)-4-amino-5-fluoro-3-[5-(4- | 852433- |
| | | Switzerland | methylpiperazin-1-yl)-1,3- | 84-2 |
| | | | dihydrobenzimidazol-2- | |
| | | | ylidene]quinolin-2-one | |
| Nintedanib | BIBF1120 | Boehringer | methyl (3Z)-3-[[4-[methyl-[2-(4- | 656247- |
| | | Ingelheim AG & | methylpiperazin-1- | 17-5 |
| | | Co. KG, Germany | yl)acetyl]amino]anilino]- | |
| | | | phenylmethylidene]-2-oxo-1H-indole- | |
| | | | 6-carboxylate | |
| Lenvatinib | E7080 | Eisai Co. Ltd., | 4-[3-chloro-4- | 417716- |
| | | Japan | (cyclopropylcarbamoylamino)phenoxy]- | 92-8 |
| | | | 7-methoxyquinoline-6-carboxamide | |
| Lucitanib | E3810 | Clovis Oncology, | 6-[7-[(1-aminocyclopropyl)methoxy]-6- | 1058137- |
| | | Inc., USA | methoxyquinolin-4-yl]oxy-N- | 23-7 |
| | | | methylnaphthalene-1-carboxamide | |
| Brivanib | BMS540215 | Bristol-Myers | (2R)-1-[4-[(4-fluoro-2-methyl-1H-indol- | 649735- |
| | | Squibb Co., USA | 5-yl)oxy]-5-methylpyrrolo[2,1- | 46-6 |
| | | | f][1,2,4]triazin-6-yl]oxypropan-2-ol | |
| ENMD-2076 | | CASI | 6-(4-methylpiperazin-1-yl)-N-(5- | 934353- |
| | | Pharmaceuticals | methyl-1H-pyrazol-3-yl)-2-[(E)-2- | 76-1 |
| | | Inc., USA | phenylethenyl]pyrimidin-4-amine | |
| BGJ398 | | Novartis AG, | 3-(2,6-dichloro-3,5-dimethoxyphenyl)- | 872511- |
| | | Switzerland | 1-[6-[4-(4-ethylpiperazin-1- | 34-7 |
| | | | yl)anilino]pyrimidin-4-yl]-1-methylurea | |
| FGF401 | | Novartis AG, | — | — |
| | | Switzerland | | |
| Lucitanib | E-3810 | Eisai Co. Ltd., | 6-[7-[(1-aminocyclopropyl)methoxy]-6- | 1058137- |
| | | Japan | methoxyquinolin-4-yl]oxy-N- | 23-7 |
| | | | methylnaphthalene-1-carboxamide | |
| PD173074 | | Pfizer, Inc., USA | 1-tert-butyl-3-[2-[4- | 219580- |
| | | | (diethylamino)butylamino]-6-(3,5- | 11-7 |
| | | | dimethoxyphenyl)pyrido[2,3- | |
| | | | d]pyrimidin-7-yl]urea | |
| SU5402 | | SUGEN, USA | 3-[4-methyl-2-[(Z)-(2-oxo-1H-indol-3- | 215543- |
| | | | ylidene)methyl]-1H-pyrrol-3- | 92-3 |
| | | | yl]propanoic acid | |
| SSR128129E | | Sanofi SA, France | sodium; 2-amino-5-(1-methoxy-2- | 848318- |
| | | | methylindolizine-3-carbonyl)benzoate | 25-2 |
| ARQ 087 | | ArQule, USA | (6R)-6-(2-fluorophenyl)-5,6-dihydro-N- | 1234356- |
| | | | [3-[2-[(2- | 69-4 |
| | | | methoxyethyl)amino]ethyl]phenyl]- | |
| | | | Benzo[h]quinazolin-2-amine | |
| LY2874455 | | Eli Lilly and Co., | 2-[4-[(E)-2-[5-[(1R)-1-(3,5- | 1254473- |
| | | USA | dichloropyridin-4-yl)ethoxy]-1H- | 64-7 |
| | | | indazol-3-yl]ethenyl]pyrazol-1- | |
| | | | yl]ethanol | |
| Debio 1347 | CH- | Debiopharm SA, | [5-amino-1-(2-methyl-3H- | 1265229- |
| | 5183284 | Switzerland | benzimidazol-5-yl)pyrazol-4-yl]-(1H- | 25-1 |
| | | | indol-2-yl)methanone | |
| TAS-120 | | Taiho Pharmaceutical | | 1448169- |
| | | Co., Ltd., Japan | | 71-8 |
| Erdafitinib | JNJ42756493 | Astex | N1-(3,5-dimethoxyphenyl)-N2- | 1346242- |
| | | Therapeutics, UK | isopropyl-N1-(3-(1-methyl-1H-pyrazol- | 81-6 |
| | | | 4-yl)quinoxalin-6-yl)ethane-1,2- | |
| | | | diamine | |
| Nintedanib | BIBF1120 | Boehringer | Methyl (3Z)-3-{[(4-{methyl[(4- | 656247- |
| | | Ingelheim AG & | methylpiperazin-1- | 17-5 |
| | | Co. KG, Germany | yl)acetyl]amino}phenyl)amino](phenyl) | |
| | | | methylidene}-2-oxo-2,3-dihydro-1H- | |
| | | | indole-6-carboxylate | |
| Orantinib | TSU68/ | Taiho | 3-[2,4-dimethyl-5-[(Z)-(2-oxo-1H-indol- | 252916- |
| | SU6668 | Pharmaceutical | 3-ylidene)methyl]-1H-pyrrol-3- | 29-3 |
| | | Co., Ltd., Japan | yl]propanoic acid | |
| FP1039 | GSK3052230 | Five Prime | | |
| | | Therapeutics Inc., | | |
| | | USA | | |
| IMC-A1 | | ImClone Systems | | |
| | | LLC, USA | | |
| PRO-001 | | ProChon Biotech | | |
| | | Ltd., USA | | |
| R3Mab | | Genentech, USA | | |
| FPA144 | | Five Prime | | |
| | | Therapeutics Inc., | | |
| | | USA | | |
| MGFR1877S | RG744 | Genentech, USA | | |
| NSC23766 | | Calbiochem, | 6-N-[2-[5-(diethylamino)pentan-2- | 1177865- |
| | | Merck KGaA, | ylamino]-6-methylpyrimidin-4-yl]-2- | 17-6 |
| | | Germany | methylquinoline-4,6-diamine | |
| EHop-016 | | — | N4-(9-Ethyl-9H-carbazol-3-yl)-N2-(3- | 1380432- |
| | | | morpholinopropyl)pyrimidine-2,4- | 32-5 |
| | | | diamine | |
| Azathioprine | Imuran | — | 6-(3-methyl-5-nitroimidazol-4- | 446-86-6 |
| | | | yl)sulfanyl-7H-purine | |
| EHT 1864 | | — | 2-(morpholin-4-ylmethyl)-5-[5-[7- | 754240- |
| | | | (trifluoromethyl)quinolin-4- | 09-0 |
| | | | yl]sulfanylpentoxy]pyran-4- | |
| | | | one; dihydrochloride | |
| SB203580 | | — | 4-[4-(4-fluorophenyl)-2-(4- | 152121- |
| | | | methylsulfinylphenyl)-1H-imidazol-5- | 47-6 |
| | | | yl]pyridine | |
| VX-702 | KVK702 | Vertex | 6-(N-carbamoyl-2,6-difluoroanilino)-2- | 479543- |
| | | Pharmaceuticals, | (2,4-difluorophenyl)pyridine-3- | 46-9 |
| | | USA | carboxamide | |
| VX-745 | | Vertex | 5-(2,6-dichlorophenyl)-2-(2,4- | 209410- |
| | | Pharmaceuticals, | difluorophenyl)sulfanylpyrimido[1,6- | 46-8 |
| | | USA | b]pyridazin-6-one | |
| Pamapimod | RO4402257 | Roche Pharma | 6-(2,4-difluorophenoxy)-2-(1,5- | 449811- |
| | | AG, Switzerland | dihydroxypentan-3-ylamino)-8- | 01-2 |
| | | | methylpyrido[2,3-d]pyrimidin-7-one | |
| Losmapimod | GW856553 | GlaxoSmithKline | 6-[5-(cyclopropylcarbamoyl)-3-fluoro- | 585543- |
| | | LLC, UK | 2-methylphenyl]-N-(2,2- | 15-3 |
| | | | dimethylpropyl)pyridine-3- | |
| | | | carboxamide | |
| Dilmapimod | SB681323 | GlaxoSmithKline | 8-(2,6-difluorophenyl)-2-((1,3- | 444606- |
| | | LLC, UK | dihydroxypropan-2-yl)amino)-4-(4- | 18-2 |
| | | | fluoro-2-methylphenyl)pyrido[2,3- | |
| | | | d]pyrimidin-7(8H)-one | |
| Doramapimod | BIRB 796 | Boehringer | 1-[5-tert-butyl-2-(4- | 285983- |
| | | Ingelheim AG & | methylphenyl)pyrazol-3-yl]-3-[4-(2- | 48-4 |
| | | Co. KG, Germany | morpholin-4-ylethoxy)naphthalen-1- | |
| | | | yl]urea | |
| BMS-582949 | PS540446 | Bristol-Myers | 4-[5-(cyclopropylcarbamoyl)-2- | 912806- |
| | | Squibb Co., USA | methylanilino]-5-methyl-N- | 16-7 |
| | | | propylpyrrolo[2,1-f][1,2,4]triazine-6- | |
| | | | carboxamide | |
| ARRY-797 | ARRY371797 | Array BioPharma | 5-(2,4-difluorophenoxy)-N-[2- | — |
| | | Inc., USA | (dimethylamino)ethyl]-1-(2- | |
| | | | methylpropyl)indazole-6-carboxamide | |
| PH797804 | | Pfizer, Inc., USA | 3-[3-bromo-4-[(2,4- | 1358027- |
| | | | difluorophenyl)methoxy]-6-methyl-2- | 80-1 |
| | | | oxopyridin-1-yl]-N,4- | |
| | | | dimethylbenzamide | |
| SCIO-469 | | Scios, Inc., USA | bismuth(2+); 2-tert-butylbenzene-1,4- | 309913- |
| | | | diol; 2-oxidobenzoate; hydrate | 83-5 |
| SD-0006 | SD06 | GlaxoSmithKline | 1-[4-[3-(4-chlorophenyl)-4-pyrimidin-4- | 271576- |
| | | LLC, UK | yl-1H-pyrazol-5-yl]piperidin-1-yl]-2- | 80-8 |
| | | | hydroxyethanone | |
| AMG-548 | | Amgen Inc., USA | 2-[[(2S)-2-Amino-3- | 864249- |
| | | | phenylpropyl]amino]-3-methyl-5-(2- | 60-5 |
| | | | naphthalenyl)-6-(4-pyridinyl)-4(3H)- | |
| | | | pyrimidinone | |
| LY2228820 | | Eli Lilly and Co., | 5-[2-tert-butyl-4-(4-fluorophenyl)-1H- | 862507- |
| | | USA | imidazol-5-yl]-3-(2,2- | 23-1 |
| | | | dimethylpropyl)imidazo[4,5-b]pyridin- | |
| | | | 2-amine; methanesulfonic acid | |
| SB239063 | | GlaxoSmithKline | 4-[4-(4-fluorophenyl)-5-(2- | 193551- |
| | | LLC, UK | methoxypyrimidin-4-yl)imidazol-1- | 21-2 |
| | | | yl]cyclohexan-1-ol | |
| Skepinone L | | — | 3-(2,4-difluoroanilino)-9-[(2R)-2,3- | 1221485- |
| | | | dihydroxypropoxy]-5,6- | 83-1 |
| | | | dihydrodibenzo[3,1-[7]annulen-11-one | |
| SB202190 | | — | 4-[4-(4-fluorophenyl)-5-pyridin-4-yl- | 152121- |
| | | | 1,3-dihydroimidazol-2- | 30-7 |
| | | | ylidene]cyclohexa-2,5-dien-1-one | |
| TAK715 | | Takeda KK, Japan | N-[4-[2-ethyl-4-(3-methylphenyl)-1,3- | 303162- |
| | | | thiazol-5-yl]pyridin-2-yl]benzamide | 79-0 |

Preferably, the viral infection to be treated by the compounds for use in the present invention are selected from the group consisting of Herpes simplex virus 1 and/or 2, Human Papilloma viruses, Ebola virus, Marburg virus, Venezuelan Equine Encephalitis virus, Chikungunya virus, Easter Equine Encephalitis virus, Western Equine Encephalitis virus, Monkey Pox virus, Corona virus, Respiratory Syncytial virus, Adenovirus, Human Rhinovirus, Influenza virus, HIV, HCV, Norovirus, Saporovirus, Cytomegalovirus (CMV), Dengue virus, West Nile virus, Yellow fever virus, Zika Virus, HSV1, HSV2, Lymphocytic Choriomeningitis virus (LCMV), HIV1, HIV2, HCV, SARS virus or MARS virus, and HBV.

In another preferred embodiment, the composition for use according to the present invention is a composition, wherein
(a) the compound is selected from the group consisting of AZD4547, BGJ398, FP1039, FPA144 and physiologically acceptable salts thereof; and
(b) the viral infection is caused by a virus selected from the group consisting of Herpes simplex virus 1 and/or 2, Human Papilloma viruses, Ebola virus, Marburg virus, Venezuelan Equine encephalitis virus, Chikungunya virus, Easter Equine Encephalitis virus, Western Equine Encephalitis virus, Monkey Pox virus, Corona virus, Respiratory Syncytial virus, Adenovirus, Human Rhinovirus, Influenza virus, HIV, HCV, Norovirus, Saporovirus, Cytomegalovirus (CMV), Dengue virus, West Nile virus, Yellow fever virus, Zika Virus, Lymphocytic Choriomeningitis virus (LCMV), and HBV.

In a further preferred embodiment, the composition for use according to the present invention is a composition, wherein the compound is selected from the group consisting of FGFR kinase inhibitors: AZD4547, Ponatinib, Dovitinib, Nintedanib, Lenvatinib, Lucitanib, Brivanib, ENMD-2076, BGJ398, FGF401, Lucitanib, PD173074, SU5402, SSR128129E, ARQ 087, LY2874455, Debio 1347, TAS-120, Erdafitinib, Nintedanib, and Orantinib; FGFR ligand traps: FP1039; and FGFR neutralizing antibodies: IMC-A1, PRO-001, R3Mab, FPA144 and MGFR1877S; and physiologically acceptable salts thereof; and the viral infection is caused by a virus selected from the group consisting of Dengue virus, HSV1, HSV2, HIV1, HIV2, HCV, Zika Virus, Lymphocytic Choriomeningitis virus (LCMV), Influenza virus, SARS virus or MARS virus.

In another preferred embodiment, the composition for use according to the present invention is a composition, wherein the compound is selected from the group consisting of AZD4547, BGJ398 and physiologically acceptable salts thereof; and the viral infection is caused by a virus selected from the group consisting of HSV1, HSV2, Lymphocytic Choriomeningitis virus (LCMV), and Zika Virus.

In a more preferred embodiment the compound for use according to the present invention is
(i) a compound for inhibiting FGFR1, FGFR2, and/or FGFR3 kinase activity, or a compound for inhibiting FGFR1, FGFR2, and/or FGFR3 kinase signaling, for the treatment of a viral disease in epithelial cells of the skin (keratinocytes), preferably an HSV1 or HSV2 infection in keratinocytes, for example, a compound selected from the group consisting of AZD4547, Ponatinib, Dovitinib, Nintedanib, Lenvatinib, Lucitanib, Brivanib, ENMD2076, BGJ398, FGF401, Lucitanib, PD173074, SU5402, SSR128129E, ARQ 087, LY2874455, Debio 1347, TAS-120, Erdafitinib, Nintedanib, Orantinib and FPA144;
preferably (ii) an FGFR ligand trap binding ligands of FGFR2b for the treatment of a viral disease affecting epithelial cells of the skin (keratinocytes), preferably an HSV1 or HSV2 infection of keratinocytes, or for the treatment of a viral disease affecting the lung, preferably an influenza virus infection of the lung, for example, a compound selected from the group consisting of AZD4547, Ponatinib, Dovitinib, Nintedanib, Lenvatinib, Lucitanib, Brivanib, ENMD-2076, BGJ398, FGF401, Lucitanib, PD173074, SU5402, SSR128129E, ARQ 087, LY2874455, Debio 1347, TAS-120, FP1039, Erdafitinib, Nintedanib, Orantinib and FPA144;
more preferably (iii) a compound for inhibiting FGFR1, FGFR2, FGFR3 and/or FGFR4 kinase activity, or a compound for inhibiting FGFR1, FGFR2, FGFR3 and/or FGFR kinase signaling for the treatment of a viral disease affecting T cells, preferably an HIV infection of T cells, for example, a compound selected from the group consisting of AZD4547, Ponatinib, Dovitinib, Nintedanib, Lenvatinib, Lucitanib, Brivanib, ENMD-2076, BGJ398, FGF401, Lucitanib, PD173074, SU5402, SSR128129E, ARQ 087, LY2874455, Debio 1347, TAS-120, Erdafitinib, Nintedanib, FP1039 and Orantinib; or
most preferably (iv) a compound for inhibiting FGFR1, FGFR2, FGFR3, and/or FGFR4 kinase activity, or a compound for inhibiting FGFR1, FGFR2, FGFR3 and/or FGFR4 kinase signaling for the treatment of a viral disease in hepatocytes, preferably an HCV or HBV infection in hepatocytes, for example, a compound selected from the group consisting of AZD4547, Ponatinib, Dovitinib, Nintedanib, Lenvatinib, Lucitanib, Brivanib, ENMD-2076, BGJ398, FGF401, Lucitanib, PD173074, SU5402, SSR128129E, ARQ 087, LY2874455, Debio 1347, TAS-120, FP1039, Erdafitinib, Nintedanib, and Orantinib.

A further preferred embodiment relates to a compound, wherein the compound is a compound for use in the present invention for inhibiting at least FGFR1 and FGFR2 kinase activity, or a compound for inhibiting at least FGFR1 and FGFR2 kinase signaling, preferably, FP1039, FPA144, AZD4547 or BGJ398, for use in the treatment of a viral disease in keratinocytes, preferably an HSV1 or HSV2 infection in keratinocytes.

In a further preferred embodiment, the composition for use according to the present invention is a pharmaceutical composition comprising at least one compound for use according to the present invention and optionally further physiologically acceptable excipients as defined above.

The compounds for use in the present invention may be administered alone or in combination with adjuvants that enhance stability, facilitate administration of pharmaceutical compositions containing them, provide increased dissolution or dispersion, increase inhibitory activity, provide adjunct therapy, and the like, including other active ingredients. The above described compounds may be physically combined with other adjuvants into a single pharmaceutical composition. Reference in this regard may be made to Cappola et al.: U.S. patent application Ser. No. 09/902,822, PCT/US 01/21860 and U.S. provisional application No. 60/313,527, each incorporated by reference herein in their entirety. The optimum percentage (w/w) of a compound or composition of the invention may vary and is within the purview of those skilled in the art. Alternatively, the compounds may be administered separately (either serially or in parallel). Separate dosing allows for greater flexibility in the dosing regimen.

As mentioned above, dosage forms of the compounds for use in the present invention include pharmaceutically acceptable carriers and adjuvants known to those of ordinary skill in the art. These carriers and adjuvants include, for example, ion exchangers, alumina, aluminium stearate, lecithin, serum proteins, buffer substances, water, salts or electrolytes and cellulose-based substances. Preferred dosage forms include, tablet, capsule, caplet, liquid, solution, suspension, emulsion, lozenges, syrup, reconstitutable powder, granule, suppository and transdermal patch. Methods for preparing such dosage forms are known (see, for example, H. C. Ansel and N. G. Popovish, Pharmaceutical Dosage Forms and Drug Delivery Systems, 5th ed., Lea and Febiger (1990)). Dosage levels and requirements are well-recognized in the art and may be selected by those of ordinary skill in the art from available methods and techniques suitable for a particular patient. In some embodiments, dosage levels range from about 5 mg-500 mg/dose for a 70 kg patient. Although one dose per day may be sufficient, up to 5 doses per day may be given. For oral doses, up to 2000 mg/day may be required. Reference in this regard may also be made to U.S. provisional application No. 60/339,249. As the skilled artisan will appreciate, lower or higher doses may be required depending on particular factors. For instance, specific doses and treatment regimens will depend on factors such as the patient's general health profile, the severity and course of the patient's disorder or disposition thereto, and the judgment of the treating physician. For example, the compounds of the present invention can be administered the same way as other virostatic medicaments.

Compounds for use in the present invention may be formulated into capsules the same way other virostatic medicaments are formulated. Each capsule may contain 25 to 500, preferably 150 to 300, more preferably 200 to 250 mg of a compound of the invention. For example, non-medicinal ingredients in capsules for the compounds of the present invention are—capsule shell: D&C yellow No. 10, FD&C blue No. 1, FD&C red No. 3, FD&C yellow No. 6, gelatin and titanium dioxide. Bottles of 100. (see also Martindale: the complete drug reference, 34th Edition, 2005, Pharmaceutical Press, p 612.)

In a further preferred embodiment, the pharmaceutical composition for use according to the present invention is for topical, oral, intravenous, intranasal, or rectal administration.

Routes of administration also include, but are not limited to intraperitoneally, intramuscularly, subcutaneously, intrasynovially, by infusion, sublingually, transdermally, or by inhalation. The preferred modes of administration are topical, oral, intravenous, intranasal, or rectal administration.

In another preferred embodiment, the pharmaceutical composition for use according to the present invention is a composition, wherein the at least one compound is selected from the group consisting of AZD4547, BGJ398 and physiologically acceptable salts thereof; the composition is for oral, intranasal, intravenous, intramuscular, intradermal, subcutaneous, intraperitoneal or topical administration, preferably topical administration; and the composition is for use in the treatment of HSV-1 inventions.

In a further preferred embodiment, the compounds or compositions defined above are used for the prophylactic or therapeutic treatment of an HSV 1 infection in keratinocytes, and the compound or composition is preferably for topical administration.

In another preferred embodiment, the compounds or compositions as defined above are for use in the prophylactic or therapeutic treatment of a viral infection in a human or animal, preferably a human, mammal or bird, more preferably a human.

Another aspect of the present invention is directed to a method for the therapeutic or prophylactic treatment of a viral disease, preferably one or more of the viral diseases listed above, comprising the steps of
(a) providing a compound or a composition as defined above; and
(b) administering the compound or composition of (a) to the subject in need thereof in a pharmaceutically effective amount, preferably by oral, intranasal, intravenous, intramuscular, intradermal, subcutaneous, intraperitoneal or topical administration, more preferably by topical administration.

Preferably the subject for treatment is selected from the group consisting of a human and an animal, preferably a human, mammal and bird, more preferably a human.

Also, the present invention relates to the use of a compound for inhibiting (i) FGFR kinase activity or (ii) a component of the FGFR kinase signaling pathway as described above in the manufacture of a medicament for the treatment of a viral infection, preferably a viral infection as defined above.

In the following, the subject-matter of the present invention and the findings of the inventors with regard to the FGFR kinase signaling pathway as well as its relevance as antiviral target are discussed with reference to the appended figures and the experimental examples presented below. It is noted that the examples relate to specific embodiments of the present invention that illustrate the present invention and which should not be construed as limiting the present invention beyond the scope of the appended claims. The following examples demonstrate directly or indirectly that compounds of different chemical structure inhibiting either (i) FGFR kinase activity or (ii) a component of the FGFR signaling pathway are suitable for treating viral infections in animals, preferably mammals, humans and birds, in general, as demonstrated for three different medically relevant viruses.

Hereafter the results of the below-described experiments are discussed.

FGFs Negatively Regulate Expression of Interferon-Stimulated Genes (ISGs) in Keratinocytes

The inventors identified a novel role of FGFR signaling in antiviral defense through control of the cell's interferon response. They also showed that ISGs are regulated by FGFR inhibition in a cell-autonomous manner in mouse and human keratinocytes. Importantly, this was not associated with alterations in interferon expression, suggesting that FGFR signaling directly modulates the tonic interferon signaling that occurs in many cell types, even in the absence of viruses (Gough et al., Immunity 36, 166-174, 2012).

FGFs Control ISG Expression in Keratinocytes Through a RAC1- and p38-Dependent Signaling Pathway

The FGF-dependent modulation of ISG expression shown by the inventors is consistent with published data showing that FGF7 suppresses the expression of a limited number of ISGs in cultured lung airway epithelial cells (Prince et al., Physiol Genomics 17, 81-89, 2011), while such a regulation has not been described for the skin or other tissues. Interestingly, mice lacking the small GTPase RAC1 in keratinocytes also showed increased expression of a similar set of ISGs (Pedersen et al., J Cell Sci 125, 5379-5390, 2012), and the inventors demonstrated that FGF7 (a type of FGF that strongly activates the FGFR2b present on keratinocytes) indeed exerts its effect on ISG expression via RAC1. In addition, p38 mitogen-activated kinase is required, since blocking of this kinase also strongly reduced the effect of FGF7 on ISG expression.

FGF7 Promotes Virus Replication in Keratinocytes

The negative effect of FGF7 on ISG expression correlated with a strong increase in the viral load of keratinocytes after infection with different viruses. This effect of FGF7 on the viral load occurred through the RAC1 signaling pathway. Consistent with the function of ISGs in the inhibition of virus infection and replication, the below described studies indicate that both pathways were promoted by FGF7, but in particular viral replication. This finding also argues against the possibility that entry of HSV-1 via an FGF receptor is responsible for the promotion of HSV-1 infection. Such a mechanism had previously been proposed for other cells (Kaner et al., Science 248, 423-431, 1990). This mechanism is further excluded by the finding that FGF7 promoted viral replication, whereas entry of HSV-1 via FGFR was blocked by recombinant FGF in the study by Kaner et al. Most importantly, the effect of FGF7 was not restricted to HSV-1. These results further argue for a general effect of FGFs on viral replication through suppression of ISG expression.

Inhibition of FGFR Signaling as a Novel Antiviral Strategy

The most relevant aspect of the below-described data is the potential use of FGFR inhibitors or of compounds that block the novel FGFR signaling pathway for the inhibition of viral infections in mammals, preferably in humans. This utility is supported by the inventors' findings that FGFR inhibition enhanced the expression of multiple antiviral genes and that FGFR or RAC1 inhibitors strongly suppressed replication of HSV-1 in cultured keratinocytes and in skin explants. Most importantly, mice lacking FGFR1 and FGFR2 in keratinocytes exhibited a strongly reduced virus load after infection of their skin with HSV-1. This result demonstrates the feasibility of FGFR inhibition for the control of virus infections in vivo. Such an approach is not limited to the skin and to HSV-1, since FGF2, another member of the FGF family, promoted hepatitis C RNA replication and production of novel particles in hepatoma cells (Van et al., Gut 65, 1015-1023, 2016). Furthermore, the below-described data show that FGF7 also increases infection of keratinocytes with LCMV and even with the important human pathogen Zika virus. Therefore, the results are relevant for the treatment of a broad spectrum of, if not all, viruses, of which many have a major impact on society.

The utility of FGFR inhibition as an antiviral strategy is particularly interesting, since FGFR kinase inhibitors and FGF ligand traps are in clinical trials for the treatment of different types of cancer (Tanner and Grose, Sem. Cell Dev. Biol 53, 216-135; Touat et al. Clin Cancer Res 21, 2684-2694, 2015). Importantly, these inhibitors were shown to be well tolerable (Tanner and Grose, Sem. Cell Dev. Biol 53, 216-135; Touat et al. Clin Cancer Res 21, 2684-2694, 2015).

Today, virostatic agents are frequently employed for the treatment of viral infections, which interfere with the viral life cycle at different stages. However, these agents are generally virus-specific and therefore susceptible to viral variation. Furthermore, they often show high toxicity. For example, current treatment options for HSV-1 involve the inhibition of the viral thymidine kinase by nucleoside analogs or by helicase-primase inhibitors. In particular the helicase-primase inhibitors show strong side effects and mutagenic potential (De et al., Curr. Opin. Infect. Dis. 28, 589-595, 2015; Piret and Boivin, Antimicrob. Agents Chemother 55, 459-472, 2011). Therefore, improved strategies are urgently needed and FGFR inhibition is a promising and fundamentally novel approach.

### FIGURES

FIG. 1 illustrates the up-regulation of ISGs in the epidermis of mice lacking FGFR1 and FGFR2 in keratinocytes.

FIGS. 1A-C show the results of qRT-PCR analysis of Irf7, Stat1, Stat2, Rsad2, OasI2 and Ma relative to Rps29 using RNA from the epidermis of FGFR1/R2 knockout (K5-R1/R2) and control (CTRL) mice at the age of 3 months (A), 9 days (B) or 5 days (C). Mean expression levels in CTRL mice were set to 1.

FIG. 1D shows confocal microscopy images of epidermal sheets from back skin of 3 month-old wild-type (WT) and K5-R1/R2 mice stained with antibodies against IRF7 (red) and K14 (green). Nuclei were counterstained with DAPI (blue). Arrows point to cells with strong expression and nuclear localization of IRF7 in K5-R1/R2 mice. Magnification bar: 20 μm.

FIG. 1E depicts the qRT-PCR analysis of Irf7, Rsad2 and Ifit1 relative to Rps29 using RNA from the epidermis of Cre positive (K5-Cre⁺) and WT mice at the age of 2 months. Mean expression levels in wild-type mice were set to 1. A representative result of two experiments is shown (A-D). All bar graphs show scatter plots and mean values. Each dot represents the expression level in an individual mouse.

FIG. 2 demonstrates that FGF signaling modulates ISGs expression in cultured keratinocytes.

FIG. 2A pertains to primary keratinocytes from CTRL and K5-R1/R2 mice (upper panel) and from K5-Cre⁺ and wild-type mice (lower panel), which were analyzed for Irf7, Stat2, and Ifit1 expression relative to Rps29 by qRT-PCR. Mean expression levels in CTRL (upper panel) or wildtype (lower panel) mice were set to 1.

FIGS. 2B-D show results from serum-starved primary and immortalized keratinocytes from wild-type mice, which were treated for 3 or 6 h with FGF7 (10 ng/ml) or vehicle (CTRL). RNA from these cells was analyzed by qRT-PCR for the expression of the indicated ISGs and of interferons relative to Rps29. Mean expression levels in CTRL cells were set to 1.

FIG. 2E are photographs of primary keratinocytes from wild-type mice that were stained with anti-IRF7 (red) and, to visualize cell junctions, with anti-ZO.1 (green) antibodies. Nuclei were stained with DAPI (blue). Arrows indicate nuclear IRF7 in CTRL samples. Images were obtained using standard wide field microscopy. Magnification bar: 50 μm.

FIG. 2F relates to serum-starved immortalized human keratinocytes (HaCaT cell line) that were treated for 6 h with FGF7, FGF10 (both 10 ng/ml) or vehicle (CTRL). RNA was analyzed by qRT-PCR for the expression of the indicated ISGs relative to RPLP0. Mean expression levels in CTRL cells were set to 1. Representative data from three experiments are shown. Scatter plot and mean values are shown in (A-D). Each dot represents a biological replicate.

FIG. 3 demonstrates that FGF7 suppresses the IFNα-mediated up-regulation of ISGs in keratinocytes at the transcriptional level.

FIG. 3A relates to primary keratinocytes from IFNAR knockout (Ifnar KO) mice and control littermate mice, which were analyzed for Ifnar, Irf7, OasI2 and Rsad2 expression relative to Rps29 by qRT-PCR. Mean expression levels in Ifnar KO samples were set to 1.

FIG. 3B pertains to primary keratinocytes from Ifnar KO mice that were treated with FGF7 or vehicle (CTRL) for 6 h and then analyzed for the expression of ISGs relative to Rps29 by qRTPCR. Mean expression levels in vehicle-treated samples from Ifnar KO cells were set to 1.

FIG. 3C pertains to immortalized mouse keratinocytes that were co-transfected with TK-Renilla and pGL4.45[luc2P/ISRE/Hygro] plasmids, starved in serum-free medium and then treated with FGF7 (10 ng/ml) and/or IFNα (1000 U/ml) for 12 h. Firefly luciferase activity was determined in cell lysates and normalized to Renilla luciferase activity (transfection control). The mean value in vehicle-treated cells (CTRL) was set to 1.

FIG. 3D shows results for serum-starved primary keratinocytes from wild-type mice that were treated with FGF7 (10 ng/ml) and/or IFNα (1000 U/ml) for 6 h and analyzed for expression of different ISGs relative Rps29 by qRT-PCR. Representative data out of two experiments are shown. All bar graphs show scatter plot and mean values. Each data point represents data obtained with cells from individual mice (A) or biological replicates (B-D).

FIGS. 3E and F pertain to confluent human primary foreskin keratinocytes and (E) or HaCaT cells (F) that were serum-starved for 24 h and treated with FGF7 (10 ng/ml) and/or IFNα (500 U/ml) for 16 h. Cells were analyzed for ISG mRNA levels by qRT-PCR relative to RPLP0. Mean expression levels in vehicle-treated (CTRL) cells were set to 1.

FIG. 4 demonstrates that FGF7 modulates ISG expression via RAC1 and p38

FIG. 4A relates to serum-starved primary keratinocytes from wild-type mice that were incubated with the p38 inhibitor SB203580 (5 μM) for 3 h, followed by incubation with FGF7 (10 ng/ml) for 6 hours. After RNA extraction, the levels of the indicated ISGs were analyzed by qRTPCR relative to Rps29. Mean expression levels in vehicle-treated cells were set to 1.

FIGS. 4B and C relate to serum-starved primary keratinocytes from wild type-mice that were incubated with the RAC1 inhibitor NSC23766 (50 μM) (A) or the FGFR1/2/3 kinase inhibitor AZD4547 (1 μM) for 3 h, followed by incubation with FGF7 (10 ng/ml) for 6 h. RNA from these cells was analyzed by qRT-PCR for different ISGs relative to Rps29. Mean expression levels in vehicle-treated cells were set to 1. Representative data out of three experiments are shown.

FIG. 4D pertains to mouse primary keratinocytes (left panel) or HaCaT cells (right panel), which had been treated with FGF7 (10 ng/ml) or vehicle for 10 min and analyzed for active RAC1 using the Active RAC1 detection kit (Cell Signaling). Activation of FGFR signaling under these conditions was verified by phosphorylation of ERK1/2 (p42/44).

FIGS. 4E and F relate to HaCaT cells that were transfected with the pRK5-myc-Rac1-Q61L expression vector or control vector (CTRL). After 24 h, cells were analyzed by Western blot using antibodies against RAC1, the Myc epitope fused to active Rac1 (MYC) and GAPDH (loading control) (E). Arrows point to the constitutively active RAC1 mutant. In addition, cells were analyzed by qRT-PCR for the indicated ISGs relative to RPLP0 (F). Mean expression levels in cells transfected with the empty vector were set to 1.

FIGS. 4G, H and I pertain to HEK293 cells that were co-transfected with TK-Renilla, pGL4.45[luc2P/ISRE/Hygro] plasmids and pRK5-Rac1-Q61L-Myc or control vectors. 24 h after transfection, cells were starved overnight in serum-free medium and analyzed by Western blot with antibodies against the Myc epitope and GAPDH (loading control) (G) or by luciferase assay (H). (I) Transfected cells were serum-starved overnight and then treated with IFNα (1000 U/ml) for 12 hours. Firefly luciferase activity was normalized to Renilla luciferase activity (transfection control). The mean value in vehicle-treated cells (CTRL) was set to 1.

FIG. 5 demonstrates that FGF7 promotes HSV-1 replication in human keratinocytes

FIGS. 5A-C relate to HaCaT cells infected with HSV-1 (MOI=0.5) after overnight serum starvation, either alone or in combination with FGF7 (10 ng/ml) or IFNα (1000U/ml). 24 h after infection, virus load was determined by qPCR for the HSV-1 Glycoprotein B (Glyc-B) gene relative to the human β-actin gene (A) or visualized by immunofluorescence staining for HSV-1 Glycoprotein D (Glyc-D) (red) (B). The Glyc-B/β-actin gene ratio in HSV-1 infected cells was set to 1. Cell nuclei were stained with DAPI (blue). Magnification bar: 200 μm. (C) Transmission microscopy images of HaCaT cells taken 48 h after HSV-1 infection. Detached cell clusters were seen in HSV-1-infected samples treated with FGF7. Magnification bar: 80 μm. Representative data out of four experiments are shown.

FIGS. 5 D & E relate to overnight-starved HaCaT cells infected with HSV-1 (MOI=0.5) alone or in combination with IFNα (1000 U/ml), IFNα and FGF7 (10 ng/ml) (D); or FGF7 at different concentrations (E). 24 h after infection, virus load was determined by qPCR for Glyc-B relative to the human β-actin gene.

FIG. 5F pertains to serum-starved HaCaT cells that were infected with HSV-1 (MOI 0.5-1). After 4 h, infected cells were washed with PBS, followed by addition of fresh serum-free culture medium containing FGF7 (10 ng/ml). 8 h later virus load was measured by qPCR for Glyc-B relative to the human β-actin gene. Representative data out of two independent experiments are shown.

FIGS. 5G-K pertains to overnight-starved HaCaT cells infected with HSV-1 (MOI=0.5) alone or in combination with FGF7, which was added at the indicated time points. 12 h after infection, cells were analyzed for Glyc-B DNA (H) and, RSAD2 and IRF7 mRNA levels by qPCR/qRT-PCR (I) or for ISG protein levels by western blotting (K). Vinculin was used as loading control.

FIG. 5J is a graph showing the mean levels of Glyc-B DNA and RSAD2 and IRF7 mRNA levels. The Glyc-B/β-actin and the ISG/RPLP0 ratios in HSV-1 infected cells were set to 1. Data are representative of two independent experiments.

FIG. 5L pertains to confluent human primary foreskin keratinocytes (HPK) that were serum-starved and then infected with HSV-1 (MOI=0.5)+/−FGF7 at the concentration indicated in the figure. 24 h after infection, virus load was measured by qPCR for Glyc-B relative to the human β-actin gene. Mean expression levels in HSV-1 infected HPK not treated with FGF7 were set to 1. Representative data out of two independent experiments are shown.

FIG. 6 illustrates that FGF7 promotes LCMV and ZIKV replication in HaCaT cells

FIG. 6A relates to HaCaT cells that were infected by LCMV (MOI=0.05 or 0.2) in the presence or absence of FGF7 or IFNα. 24h after infection cells were analyzed for LCMV Nucleoprotein (NP) expression using flow cytometry. The percentage of LCMV positive cells was set to 1. Each data point represents one biological replicate.

FIGS. 6B and C relate to HaCaT cells that were serum starved overnight and then infected with the ZIKV strains Uganda (strain 976) (MOI=0.1) or French-Polynesia (PF13/251013-18) (MOI˜20). 2h after infection cells were treated with 20 ng/ml FGF7 or left untreated (CTRL). Culture medium+/−FGF7 was changed every day. 48h post infection cells were analyzed by immunofluorescence using a Flavivirus group-specific antibody (4G2) detecting the ZIKV envelope protein (ZIKV-Env) (B). Magnification bar in (B): 40 μm. Alternatively, cells were analyzed for ZIKV mRNA levels by qRT-PCR relative to RPL27 (C). Mean expression levels in vehicle-treated cells were set to 1. Representative out of four independent experiments are shown.

FIGS. 6D and E pertain to HaCaT cells that were infected with the ZIKV strain French-Polynesia (PF13/251013-18) (MOI˜20) after overnight starvation, and treated or not with 20 ng/ml FGF7. 72h post infection cells were harvested and analyzed by qRT-PCR for OAS1 and MxA relative to RPL27. For FIG. 6D expression levels in uninfected cells (CTRL) were set to 1. For FIG. 6E expression levels of infected cells not treated with FGF7 were set to 1. Representative data out of four independent experiments are shown.

FIG. 7 illustrates that FGF7 promotes HSV-1 replication in vitro and ex vivo via FGFR kinase activity and RAC1

FIGS. 7A-C relate to serum-starved HaCaT cells that were infected with HSV-1 in the presence or absence of FGF7 (10 ng/ml), NSC23766 (20 μM), the FGFR kinase inhibitors AZD4547 (1 μM) and BGJ398 (3.5 μM) (A) or IFNα (1000 U/ml) (B). After 16 h, the viral load was analyzed by qPCR for Glyc-B relative to the human β-actin gene (A, B) and by Glyc-D immunofluorescence staining (red) (C, left). Magnification bar: 500 μm. The Glyc-D positive area was quantified using digital pixel measurements (C, right). A representative out of two experiments is shown.

FIG. 7D pertains to epidermal sheets from tail skin of wild-type mice that were infected ex vivo with HSV-1 (MOI=2) in the presence or absence of IFNα, FGF7, NSC23766 or AZD4547 and analyzed by immunofluorescence staining 48 h after infection with antibodies against Glyc-D (red). A strong increase in infected cells was seen after FGF7 treatment. Cell nuclei were stained using DAPI (blue). Magnification bar: 800 μm. Values obtained for HSV-1 infected cells in (A-C) without additional treatment were set to 1.

FIG. 7E pertains to mice lacking FGFR1 and FGFR2 in keratinocytes (K5-R1/R2) mice and age- and sex-matched control mice that were infected with 50 μl HSV1 (MOI=10) (4 injections into the back skin of each mouse). 48h after infection, the injected skin was removed and the amount of viral DNA encoding the immediate-early protein ICP0 was determined and normalized to the host gene Tbx15. The ICP0/Tbx15 ratio in infected skin of control mice was set to 1. Scatter plots and mean values are shown in all bar graphs. Each data point represents one biological replicate (A-D) or data from a single HSV-1 infection spot (E).

FIG. 8 is Table 1, which shows ISGs that are overexpressed in the epidermis of K5-R1/R2 mice compared to control mice. Expression of all listed genes is significantly and more than 2-fold regulated.

### EXAMPLES

### Example 1: Materials and Methods

Antibodies, Recombinant Proteins and Chemical Compounds

The following antibodies were used for Western blotting and/or immunofluorescence staining: anti-IRF7 (sc-9083, Santa Cruz, Calif.), anti-RSAD2 (13996, Cell Signaling), anti-glyceraldehyde 3-phosphate dehydrogenase (GAPDH) (5G4, HyTest, Turku, Finland), anti-Zona Occludens 1 (ZO.1) (339100, Invitrogen, Carlsbad, Calif.), anti-Keratin 14 (K14) (PRB-155P, BioLegend, San Diego, Calif.), anti-Myc Tag clone 9E10 (MA1-980, Thermo Fisher, Waltham, Mass.), anti-human influenza virus hemagglutinin (HA) (H6908, Sigma, Munich, Germany), anti-Vinculin (v4505, Sigma), anti-Rac1 (05-389, Merck-Millipore, Darmstadt, Germany), anti-HSV-1 glycoprotein D (Glyc-D) (ab27586, Abcam, Cambridge, UK), anti-LCMV nucleoprotein (clone VL-4) (kindly provided by Prof. Rolf M. Zinkernagel, University of Zurich), AF488-conjugated anti-mouse IgG (A-11001, Thermo Fisher), anti-lamin A (sc-6214, Santa Cruz, Calif.), anti-Phosphop-44/42 MAPK (Erk1/2) (9101, Cell Signaling), anti-p44/42 MAPK (Erk1/2) (9102, Cell Signaling), anti-HSV-1 glycoprotein D (Glyc-D) (ab27586, Abcam, Cambridge, UK), anti-Flavivirus group antigen antibody, clone D1-4G2-4-15 (MAB10216, Merck-Millipore), AF555-conjugated anti-mouse IgG (A-21422), AF488-conjugated anti-mouse IgG (A-11001) and AF555-conjugated anti-rabbit IgG (A-21428, all from Thermo Fisher).

The following recombinant proteins and chemical inhibitors were used: human FGF7 (100-19, PeproTech Inc., Rocky Hill, N.J.), human FGF10 (100-26, PeproTech Inc.), RAC inhibitor NSC23766 (S8031, Selleckchem, Houston, Tex.), FGFR1/2/3 inhibitors AZD4547 (S2801, Selleckchem) and BGJ398 (NVP-BGJ398) (S2183, Selleckchem), p38 MAPK inhibitor SB203580 (S1076, Selleckchem).

Genetically Modified Mice and Infection of Mice with HSV-1

Mice lacking Fgfr1 and Fgfr2 in keratinocytes (K5-R1/R2 mice) had previously been described (Yang et al., J. Cell Biol. 188, 935-952, 2010). All mice were in C57BL/6 genetic background. They were housed under specific pathogen-free (SPF) conditions and maintained according to Swiss animal protection guidelines. For HSV-1 infection the back of mice was shaved, and 24 h later each mouse received 4 subcutaneous injections of 50 μl HSV1 (MOI=10) on the back. 48 h after HSV-1 injection, the skin was removed and the amount of viral immediate-early protein ICP0 DNA (Primers: 5′-ATA AGT TAG CCC TGG CCC CGA-3′, SEQ ID NO: 1, and 5′-GCT GCG TCT CGC TCC G-3′, SEQ ID NO: 2) was determined by qPCR and normalized to the host Tbx15 DNA (Primers: 5′-TCC CCC TTC TCT TGT GTC AG-3′, SEQ ID NO: 3 and 5′-CGG AAG CAA GTC TCA GAT CC-3′, SEQ ID NO: 4). All procedures with mice had been approved by the veterinary authorities of Zurich, Switzerland (Kantonales Veterinaramt Zurich).

Cell Culture

Primary mouse keratinocytes were isolated from neonates as described (Yang et al., 2010 J. Cell Biol. 188, 935-952, 2010) and cultured for 3 days in a 7:5 mixture of keratinocyte serum-free medium (Life Technologies, Carlsbad, Calif.) supplemented with 10 ng/ml epidermal growth factor, 10⁻¹⁰ M cholera toxin and 100 U/ml penicillin/100 μg/ml streptomycin (Sigma) and of keratinocyte medium. Plates were coated with collagen IV (Sigma) prior to seeding of the cells. Spontaneously immortalized keratinocytes from wild-type mice had previously been described (Yang et al., 2010, J. Cell Biol. 188, 935-952, 2010).

For treatment of primary or immortalized mouse keratinocytes with FGF7 (10 ng/ml), FGF10 (10 ng/ml) and/or IFNα (1000 U/ml) cells were grown to confluency, starved overnight in keratinocyte serum-free medium, treated for the indicated time points, and harvested. For the treatment with NSC23766 (20 μM), AZD4547 (1 μM), SB203580 (5 μM) or vehicle (DMSO), cells were pre-incubated with the indicated inhibitors for 3 h at 37° C., prior to treatment with FGF7 (10 ng/ml) and harvested 6 h later.

Human primary foreskin keratinocytes were seeded in keratinocyte serum free medium (Gibco BRL, Paisley, UK), supplemented with epidermal growth factor and bovine pituitary extract (Gibco BRL). Cells were used for experiments between passage 3 and 5.

The human HaCaT keratinocyte cell line was cultured in DMEM (Sigma) supplemented with 10% FCS (Thermo Fisher). For treatment of human primary or HaCaT keratinocytes with FGF7, FGF10 or IFNα, cells were grown to confluency and maintained in the absence of serum or purified growth factors for 24 h prior to addition of 10 ng/ml FGF7 or FGF10 and/or 500 U/ml IFNα. Human embryonic kidney cells (HEK) 293 cells (85120602, Sigma) were cultured in DMEM/10% FCS and maintained in serum-free DMEM for 12 h prior the addition of 500 U/ml IFN a.

Separation of Dermis from Epidermis of Mouse Back Skin

Mouse epidermis was separated from dermis by heat shock treatment (30 sec at 55-60° C. followed by 1 min at 4° C., both in PBS), or by incubation for 50-60 min at 37° C. in 0.143% dispase (17105-041, Life Technologies)/DMEM or by incubation in 0.8% trypsin (27250-018, Life Technologies)/DMEM for 15-30 min at 37° C. For dispase and trypsin treatment the subcutaneous fat was gently scraped off with a scalpel prior to incubation.

RNA Isolation and qRT-PCR

Total RNA from isolated epidermis of mice or from total skin was purified with Trizol, followed by additional purification with the RNeasy Mini Kit, including on-column DNase treatment (Qiagen, Hilden, Germany). Total RNA from cultured cells was directly extracted with the RNeasy Mini Kit. cDNA was synthesized using the iScript kit (Bio-Rad Laboratories, Berkeley, Calif.). Relative gene expression was determined using the Roche LightCycler 480 SYBR Green system (Roche, Rotkreuz, Switzerland).

Expression of the following mouse genes was analyzed by qRT-PCR using the primers listed below: Ma, Irf7, OasI2, Rps29, Stat1, Rsad2, Stat2, Ifnα, Ifnβ, Il28a: (primers, forward and reverse):

Ifit1:
SEQ ID NO: 5
5′-AGC AAC CAT GGG AGA GAA TGC-3′,;
SEQ ID NO: 6
5′-CCT TTC AGG TGC CTC ACG TA-3′,;
Irf7:
SEQ ID NO: 7
5′-AGC TTG GAT CTA CTG TGC GC-3′,;
SEQ ID NO: 8
5′-GGG TTC CTC GTA AAC ACG GT-3′,;
Oasl2:
SEQ ID NO: 9
5′-TGC CTG GGA GAG AAT CGA AG-3′,;
SEQ ID NO: 10
5′-AGC CTC CCT TCA CCA CCT TA-3′,;
Rps29:
SEQ ID NO: 11
5′-GGT CAC CAG CAG CTC TAC TG-3′,;
SEQ ID NO: 12
5′-GTC CAA CTT AAT GAA GCC TAT GTC C-3′,;
Stat1:
SEQ ID NO: 13
5′-GGA TCG CTT GCC CAA CTC T-3′,;
SEQ ID NO: 14
5′-GCA GAG CTG AAA CGA CCT AGA-3′,;
Rsad2:
SEQ ID NO: 15
5′-GGA GGT GGT GCA GGG ATT AC-3′,;
SEQ ID NO: 16
5′-GGA AAA CCT TCC AGC GCA CA-3′,;
Stat2:
SEQ ID NO: 17
5′-CTT TTG CAA GCG AGA GAG CC-3′,;
SEQ ID NO: 18
5′-TGA AGC GCA GTA GGA AGG TG-3′,;
Ifnα:
SEQ ID NO: 19
5′-TCT CTC CAC ACT TTG TCT CAC AC-3′,;
SEQ ID NO: 20
5′-ACA GTC CAG AGA GCC ATC AAC C-3′,;
Ifnβ:
SEQ ID NO: 21
5′-AAG ATC TCT GCT CGG ACC AC-3′,;
SEQ ID NO: 22
5′-TGG GAG ATG TCC TCA ACT GC-3′,;
Il28a (Ifn λ2):
SEQ ID NO: 23
5′-GCAGACCTGTACACAGCTTCA-3′,;
SEQ ID NO: 24
5′-CAGGTTGGAGGTGACAGAGG-3′,;

Expression of the following human genes was analyzed by qRT-PCR using the primers listed below: IFIT1, IRF7, RPLP0, RSAD2, STAT1, STAT2, OAS2, OAS1, MxA, and RPL27: (primers, forward and reverse):

IFIT1:
SEQ ID NO: 25
5′-AGC TTA CAC CAT TGG CTG CT-3′,;
SEQ ID NO: 26
CCA TTT GTA CTC ATG GTT GCT GT-3′,;
IRF7:
SEQ ID NO: 27
5′-AGC TGT GCT GGC GAG AAG-3′,;
SEQ ID NO: 28
CTC TCC AGG AGC CTT GGT TG-3′,;
RPLP0 (36B4):
SEQ ID NO: 29
5′-CCA CAT TGT CTG CTC CCA CA-3′,;
SEQ ID NO: 30
5′-GAA GAC AGG GCG ACC TGG AA-3′,;
RSAD2:
SEQ ID NO: 31
5′-GCT GCT AGC TAC CAA GAG GAG-3′,;
SEQ ID NO: 32
ATC TTC TCC ATA CCA GCT TCC-3′,;
STAT1:
SEQ ID NO: 33
5′-AAA GGA AGC ACC AGA GCC AAT-3′,;
SEQ ID NO: 34
TCC GAG ACA CCT CGT CAA AC-3′,;
STAT2:
SEQ ID NO: 35
5′-GGA TCC TAC CCA GTT GGC TG-3′,;
SEQ ID NO: 36
GAG GGT GTC TTC CCT TTG GC-3′,;
OAS2:
SEQ ID NO: 37
5′-GGG CTA TTT CCA GAC AAC GC-3′,;
SEQ ID NO: 38
GAA AAC CAG GCC TGT GAT CTT GG-3′,;
OAS1:
SEQ ID NO: 39
5′-TTC CTC CCT GCC ATT CAT CC-3′,;
SEQ ID NO: 40
5′-TCC AGA AAC CCT CGA TTG TGA-3′,;
MxA:
SEQ ID NO: 41
5′-ACC TAC AGC TGG CTC CTG AA-3′,;
SEQ ID NO: 42
5′-GCA CTC AAG TCG TCA GTC CA-3′,;
RPL27:
SEQ ID NO: 43
5′-AAA GCT GTC ATC GTG AAG AAC-3′,;
SEQ ID NO: 44
5′-GCT GCT ACT TTG CGG GGG TAG-3′,;

Immunofluorescence Staining

Frozen sections from mouse back skin were fixed with cold methanol, and unspecific binding sites were blocked with PBS/2% bovine serum albumin (BSA) (Sigma)/1% fish skin gelatin (Sigma)/0.05% Triton X-100 (Carl Roth GmbH, Karlsruhe, Germany) for 2 h at room temperature. Samples were then incubated overnight at 4° C. with anti-IRF7 or anti-K14 antibodies diluted in the same buffer. After three washes with 1×PBS/0.1% Tween 20 (Carl Roth GmbH), slides were incubated at room temperature (RT) for 4 h with secondary antibodies (AF555-conjugated anti-rabbit IgG and AF488-conjugated anti-mouse IgG) and DAPI (4′,6-diamidino-2-phenylindole dihydrochloride) (Sigma) as counter-stain, washed again and mounted with Mowiol (Hoechst, Frankfurt, Germany). Stained sections were photographed with a Leica SP1-2 confocal microscope equipped with a 63×0.6-1.32 NA (Iris) PL Apo Oil objective. For data acquisition the Leica Confocal Software (Leica, Wetzlar, Germany) was used. For immunofluorescence staining of cultured cells, they were washed with PBS and either fixed for 5 min with cold methanol for staining with antibodies against ZO-1 and IRF7, or with 4% paraformaldehyde (PFA) (Sigma) for 20 min at RT for Glyc-D staining. PFA-fixed cells were then incubated for 10 min with 0.5% Triton X-100 in PBS. After 1 h blocking in PBS containing 2% BSA, cells were stained with the primary antibodies for 1 h in the same blocking buffer. After three washes with PBS, cells were incubated with the secondary antibodies (AF555-conjugated anti-mouse IgG, AF488-conjugated anti-mouse IgG and AF555-conjugated anti-rabbit IgG) and DAPI. Stained cells were photographed with a Zeiss Imager.A1 microscope equipped with an Axiocam MRm camera and EC Plan-Neofluar objectives (10×/0.3, 20×/0.5). For data acquisition the Axiovision 4.6 software was used (all from Carl Zeiss Inc., Jena, Germany).

Preparation of Cytosolic and Nuclear Lysates

For nuclear/cytoplasmic fractionation cells were lysed in 0.1% NP-40 (Calbiochem, San Diego, Calif.) in PBS containing Complete Protease and Phosphatase Inhibitor Cocktails (04693116001 and 04906845001, Roche). After full speed centrifugation, the cytoplasmic fraction was removed and the pellet representing the nuclear fraction was washed 5 times with lysis buffer. Nuclear pellets and cytoplasmic fractions were prepared for SDS-PAGE by adding Laemmli sample buffer and boiled at 95° C. for 5 minutes.

Preparation of Protein Lysates and Western Blotting

Cells were harvested in T-PER tissue protein extraction reagent (Pierce, Rockford, Ill.) containing Complete Protease Inhibitor Cocktail (Roche). Lysates were cleared by centrifugation (13,000 rpm, 30 min, 4° C.), snap frozen, and stored at −80° C. The protein concentration was determined using the BCA Protein assay (Pierce). Proteins were separated using SDS-PAGE and transferred onto nitrocellulose membranes. Membranes were then incubated with the primary antibodies. After washing, antibody-bound proteins were detected with horseradish peroxidase coupled antibodies against goat-IgG (Sigma), rabbit-IgG, or mouse IgG (both from Promega, Madison, Wis.).

Cell Transfection

The expression vector pRK5-myc-Rac1-Q61L was obtained from Prof. Giorgio Scita (Firc Institute of Molecular Oncology, Milan, Italy). The expression vector p3×FLAG-MLK1 was obtained from the non-profit plasmid repository Addgene (cat. 11978, Cambridge, Mass.). Immortalized mouse keratinocytes were seeded on 6-well plates (600′000/well), incubated for 24 h and transfected with the expression vectors or empty control vectors using Lipofectamine 2000 reagent (Invitrogen) as described by the manufacturer. After 24 h, cells were lysed in Trizol and T-PER buffer for subsequent qRT-PCR or Western blot analysis, respectively.

Luciferase Assay

Mouse keratinocytes and HEK 293 cells were transfected with the expression vector for TK-Renilla and the expression vector pGL4.45[luc2P/ISRE/Hygro] (Promega). The latter contains five copies of an ISRE that drives expression of the luciferase reporter gene. Cells were seeded into 12-well plates, cultured for 24 h, and transfected using Lipofectamine 2000 (Qiagen). They were then starved in serum-free medium, treated with FGF7 (10 ng/ml) and/or IFNα (1000 U/ml) for 12 h, lysed and analyzed using a dual-luciferase assay system (Promega) as described by the manufacturer. Relative light units were measured in a GloMax 96 microplate luminometer with dual injectors (Promega).

HSV-1 Production and Cell Infection

HSV-1 viruses were produced as described (Strittmatter et al. J. Invest. Dermatol. 136, 610-620, 2016). Sub-confluent HaCaT cells were starved overnight in serum-free medium and then incubated with HSV-1 (MOI=0.5). Where indicated, infection was preceded by treatment with NSC23766 20 μM, AZD4547 1 μM, BGJ398 3.5 μM or DMSO for 3 h before treatment with FGF7 (10 ng/ml). Infected cells were left for 16 h before the assessment of viral load (see below).

Isolation of Genomic Human DNA and of Viral DNA from HSV-1 Infected Cells

Genomic and viral DNA was isolated from infected HaCaT cells using the HotSHOT genomic DNA preparation method (Truett et al., Biotechniques 29, 52-54, 2000) modified according to Strittmatter et al. (J. Invest. Dermatol. 136, 610-620, 2016). Briefly, supernatants of infected cells were removed, and 200 μl of alkaline lysis buffer (25 mM NaOH, 0.2 mM EDTA) per plate of a 6-well plate were added to the remaining cells. Cells were scratched off from the dish and the lysates were incubated in 1.5 ml Eppendorf tubes for 30 min at 95° C. Tubes were cooled down to 4° C., and 200 μl of neutralization buffer (Tris-HCl 40 mM) were added before the samples were centrifuged (13000 rpm, 10 min, 4° C.) and the DNA concentration in the supernatant determined. Samples were used for qPCR to measure HSV-1 replication/virus load. Primers for amplification of the genomic DNA for β-actin (5′-TAC TCC TGC TTG CTG ATC CAC-3′, SEQ ID NO: 45; and 5′-TGT GTG GGG AGC TGT CAC AT-3′, SEQ ID NO: 46) and viral glycoprotein B (GLYC-B) (5′-CGC ATC AAG ACC ACC TCC TC-3′, SEQ ID NO: 47; and 5′-GCT CGC ACC ACG CGA-3′, SEQ ID NO: 48) were used.

Ex Vivo HSV-1 Infection

HSV-1 infection of epidermal sheets from mouse tails was performed as previously described (Rahn et al., J. Invest. Dermatol 135, 3009-3016, 2015). Briefly, skin was removed from the tails of 3 month-old mice, followed by separation of the epidermis from the dermis by dispase treatment (5 mg/ml). After floating the epidermal sheets on serum-free DMEM overnight, they were incubated with HSV-1 alone (MOI=2) or in combination with FGF7 (15 ng/ml), IFNα (1000 U/ml) and/or NSC23766 (50 μM) or AZD4547 (1 μM) for 48 h and subsequently fixed in 4% PFA for 1 h at RT. The sheets were then incubated in blocking solution (PBS 2% BSA/1% fish skin gelatin/0.05% Triton Tx-100) for 2 h at RT and stained overnight at 4° C. with an antibody against Glyc-D diluted in blocking solution. After 3 washes in PBS, epidermal sheets were incubated for 4 h with AF555-conjugated anti-mouse IgG and DAPI diluted in PBS 0.05% Triton X-100 at RT and successively mounted with their basal side on top of a specimen slide, embedded in Mowiol and covered with coverslips. Stained samples were photographed as described for immunofluorescence analysis of cultured cells.

LCMV Infection and Flow Cytometry Analysis

Sub-confluent HaCaT cells were starved overnight in serum-free medium and incubated overnight at 37° C. with the LCMV (MOI 0.05 and 0.2). Afterwards, cells were detached from the 12-well plate by incubation in 1% trypsin and fixed/permeabilized in 500 μl 2×FACS Lyse (Becton Dickinson, Franklin Lakes, N.J.) with 0.05% Tween 20 for 10 min at room temperature. After washing, intracellular staining was performed for 30 min at room temperature using the LCMV nucleoprotein-specific antibody VL-4. After an additional washing step they were resuspended in PBS containing 1% PFA. Flow cytometry analysis was performed using an LSRII flow cytometer (Becton Dickinson). Raw data were analyzed using FlowJo software (Tree Star Inc, Ashland, Oreg.).

ZIKV Infection

HaCaT cells were seeded on a 4-well tissue chamber on a PCA slide (5×10⁴/chamber). After overnight serum starvation, they were infected with the ZIKV strains Uganda (strain 976) (MOI=0.1) or French-Polynesia (PF13/251013-18) (MOI˜20). 2h post infection cells were treated with FGF7 or left untreated. Culture media+/−FGF7 was changed every day. 48 hours post-infection cells were either analyzed by immunofluorescence using a Flavivirus group-specific antibody (4G2) detecting the ZIKV envelope protein (ZIKV-Env) or harvested and analyzed for ZIKV expression levels by qRT-PCR relative to human RPL27 (ZIKV primers: 5′-AGA TCC CGG CTG AAA CAC TG-3′, SEQ ID NO: 49; 5′-TTG CAA GGT CCA TCT GTC CC-3′, SEQ ID NO: 50). To investigate the effect of ZIKV and FGF7 on ISG expression, HaCaT cells were seeded in 6-well plates. After overnight serum starvation, confluent cells were infected with the ZIKV strain French-Polynesia (PF13/251013-18) (MOI˜20) in the presence or absence of FGF7 (20 ng/ml). 72 h post-infection cells were harvested and analyzed for OAS1 and MxA by qRT-PCR relative to RPL27.

Gene Expression Profiling and Bioinformatics Analysis

Epidermis from 9 K5-R1/R2 and 9 control mice at postnatal day 18 was separated from the dermis as previously described (Yang et al., J. Cell Biol. 188, 935-952, 2010) and used for RNA isolation. RNA samples from three mice per genotype were pooled and subjected to Affymetrix microarray hybridization (N=3 pools per genotype). Genes, which were significantly and more than 2-fold up- or down-regulated in K5-R1/R2 compared to control mice were analyzed by Ingenuity Pathway Analysis (Qiagen).

Statistical Analysis

Statistical analysis was performed using the PRISM software (Graph Pad Software Inc., San Diego, Calif.). Mann-Whitney U test for non-Gaussian distribution was used for experiments examining differences between two groups. *P≤0.05, **P≤0.01, ***P≤0.001.

### Example 2: Loss of FGF Signaling Enhances Expression of Interferon-Stimulated Genes (ISGs) in Keratinocytes

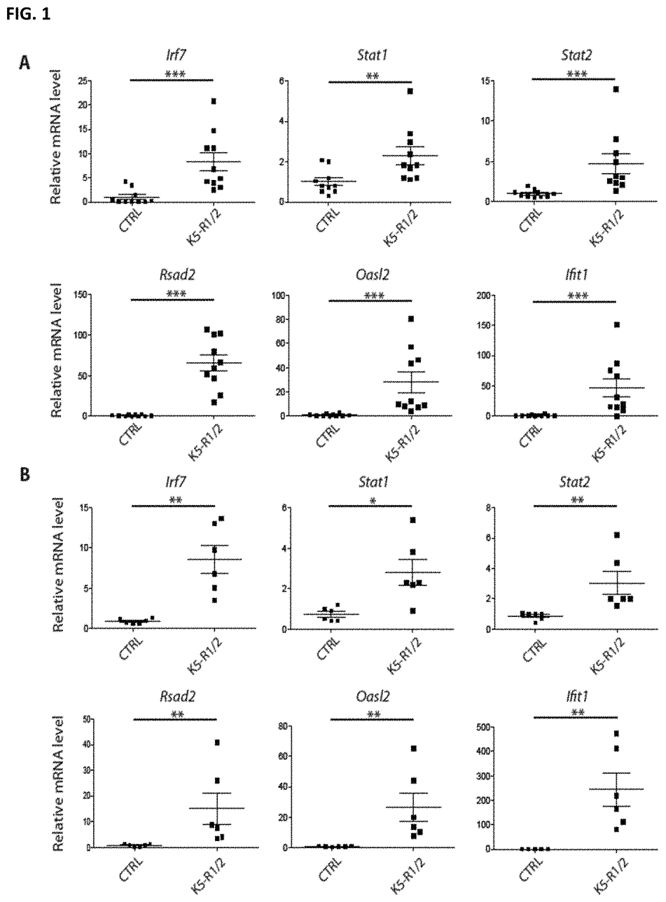

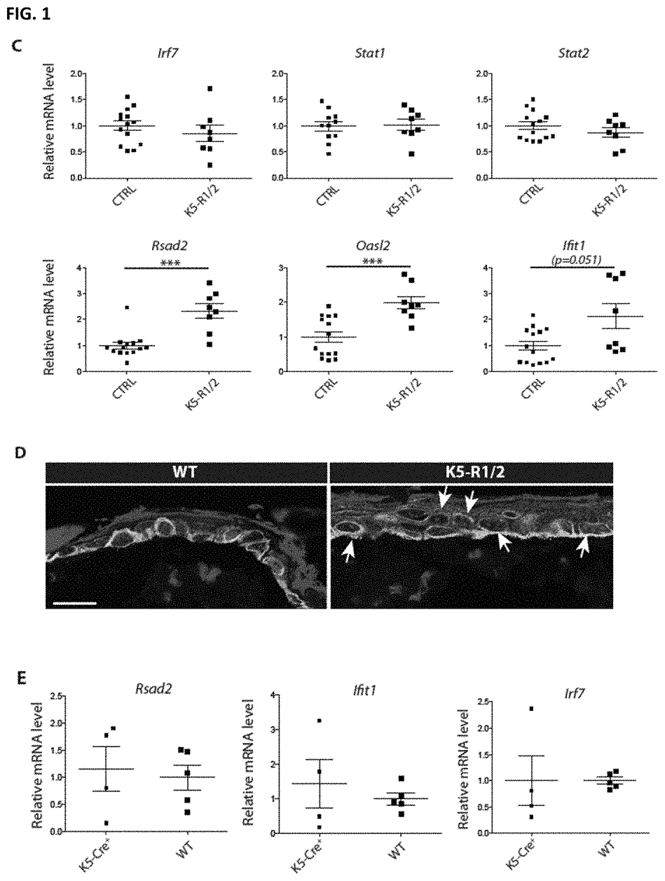

mRNA expression profiling of epidermal sheets from mice lacking FGF receptors 1 and 2 in keratinocytes (K5-R1/R2 mice) and control animals (floxed R1/R2 mice without Cre) (Yang et al., J. Cell Biol. 188, 935-952, 2010) at the age of P18 (18 days after birth) showed that many genes that are upregulated in K5-R1/R2 mice are involved in the type I interferon (IFN) response (Table 1, see FIG. 8). This was confirmed by quantitative PCR (qRT-PCR) using epidermal RNA from K5-R1/-R2 and control mice at the age of 3 months (FIG. 1A). Up-regulation of some ISGs was already significant at P5 when the Cre-mediated knockout was almost complete and very robust at P9 (FIG. 1B, C). These results were confirmed by immunofluorescence staining of IRF7 (FIG. 1D). ISG mRNA levels were similar between epidermis from Cre positive (K5-Cre) and wild-type mice (FIG. 1E), demonstrating that Cre does not affect expression of ISGs in keratinocytes.

### Example 3—FGFR Signaling Controls ISG Expression in Cultured Keratinocytes

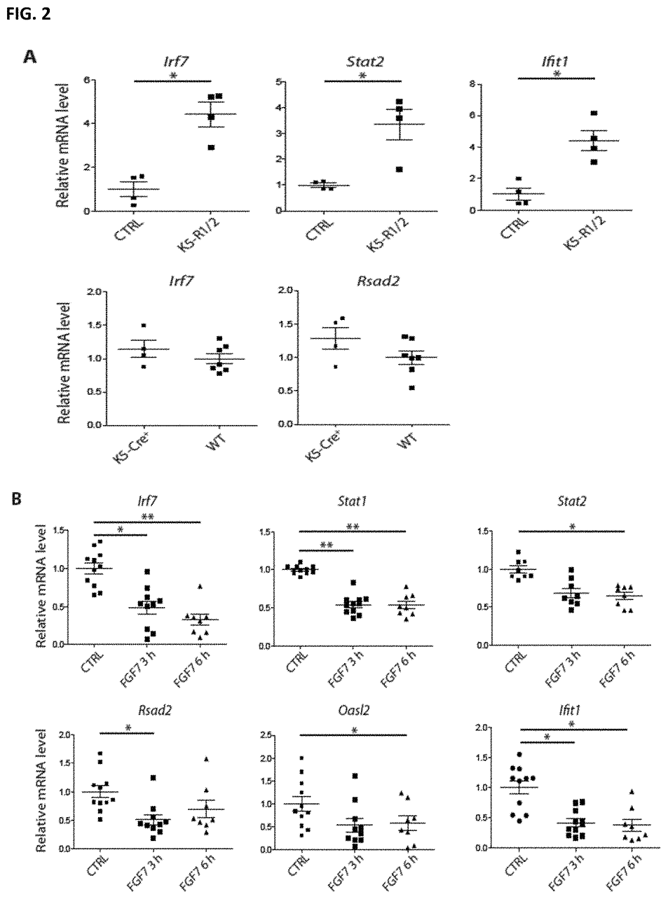

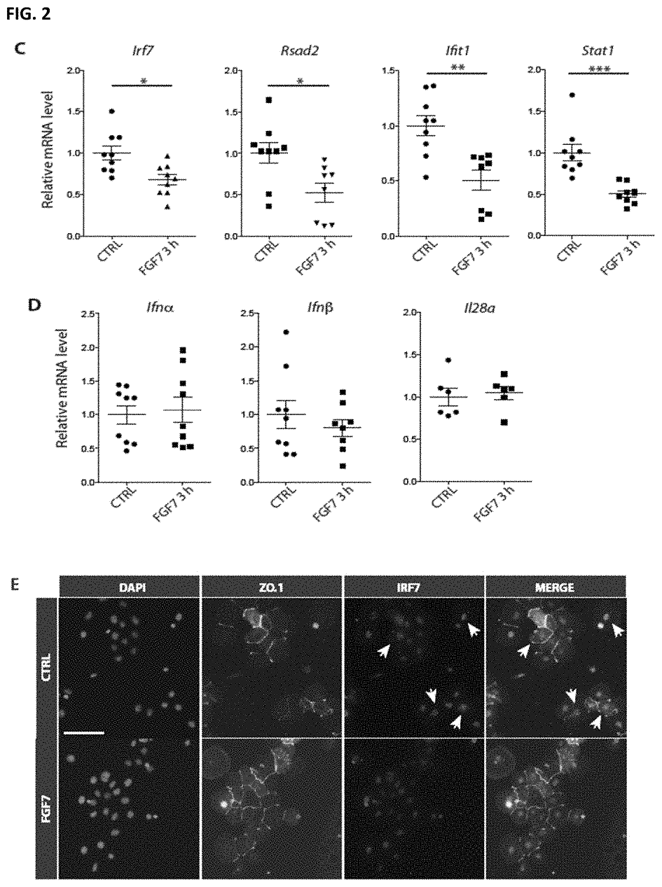

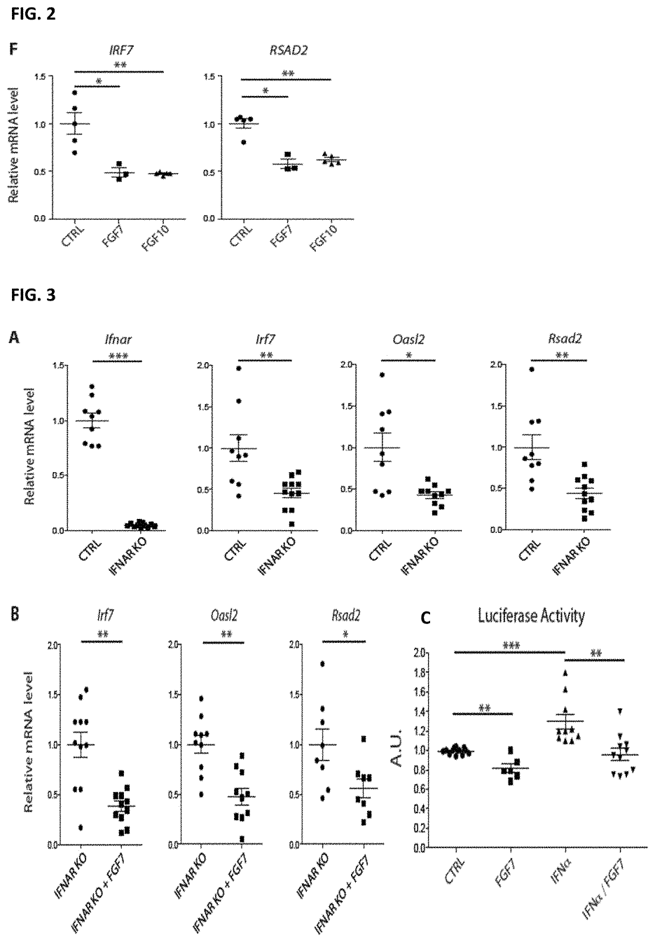

Expression levels of Irf7, Stat2, and Ifit1 were also higher in FGFR1/2-deficient cells in vitro (FIG. 2A, up), and there was no difference between K5-Cre and wild-type mice (FIG. 2A, down). This finding suggested that FGFR signaling directly regulates ISG expression.

This was confirmed with FGF7-treated primary and spontaneously immortalized keratinocytes from wild-type mice. FGF7 treatment strongly suppressed ISG expression in both cell types (FIG. 2B, C). However, expression of the different subtypes of IFN-alpha (Ifnα), and of Ifnβ and Il28a (Ifnλ2) (FIG. 2D) was not reduced, and expression of Ifnγ was hardly detectable by qRT-PCR. Therefore, the down-regulation of ISGs does not result from an FGF7-mediated decrease in IFN expression. Immunofluorescence analysis confirmed the FGF-mediated downregulation of IRF7 at the protein level (FIG. 2E). FGF10, which also activates the FGFR2b variant expressed by keratinocytes and also FGFR1b, suppressed ISG expression to a similar extent as FGF7 (FIG. 2F).

### Example 4—Suppression of ISG Expression is Independent of Interferon Receptors

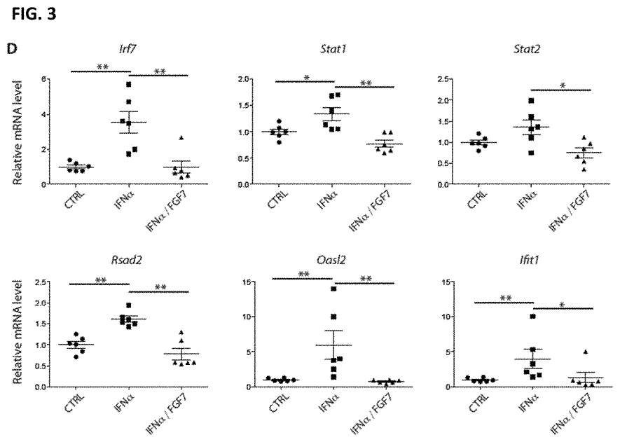

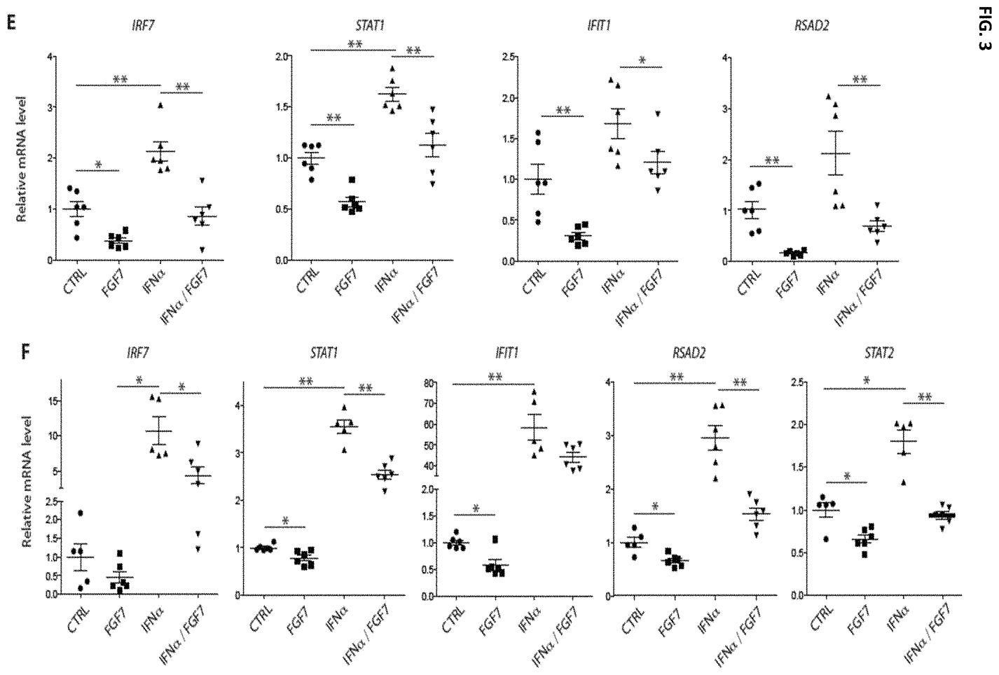

Expression of different ISGs was strongly reduced in primary keratinocytes from INFα receptor (IFNAR) knockout mice (FIG. 3A), most likely due to inhibition of the tonic IFN signaling that results from continuous production of small amounts of IFNs by keratinocytes. However, FGF7 still suppressed expression of ISGs in IFNAR-deficient cells with a similar efficiency as in wild-type keratinocytes (FIG. 3B), suggesting that FGFs regulate ISG expression downstream of the IFN receptors.

To determine if FGFs regulate ISG expression at the transcriptional level, immortalized mouse keratinocytes were transfected with a reporter plasmid in which luciferase expression is under control of an interferon-stimulated response element (ISRE). The ISRE controls the expression of most of ISGs. IFNα stimulation of the transfected keratinocytes indeed caused a significant increase in luciferase activity, which was counteracted by FGF7 (FIG. 3C). Consistent with this finding, the increase in ISG expression seen in both mouse and human keratinocytes in response to IFNα treatment was reduced in the presence of FGF7 (FIG. 3D-F). This experiment also shows that the effect of FGF7 on ISG expression is not restricted to mouse cells, but also occurs in human cells.

### Example 5—FGFs Control ISG Gene Expression in Keratinocytes Via FGFR-RAC1 Signaling

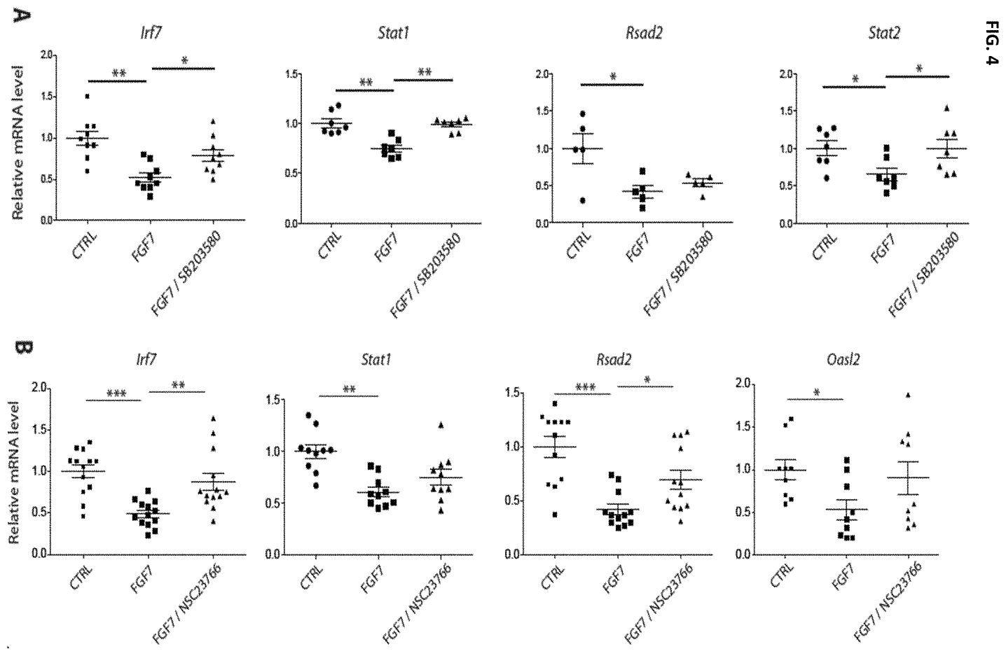

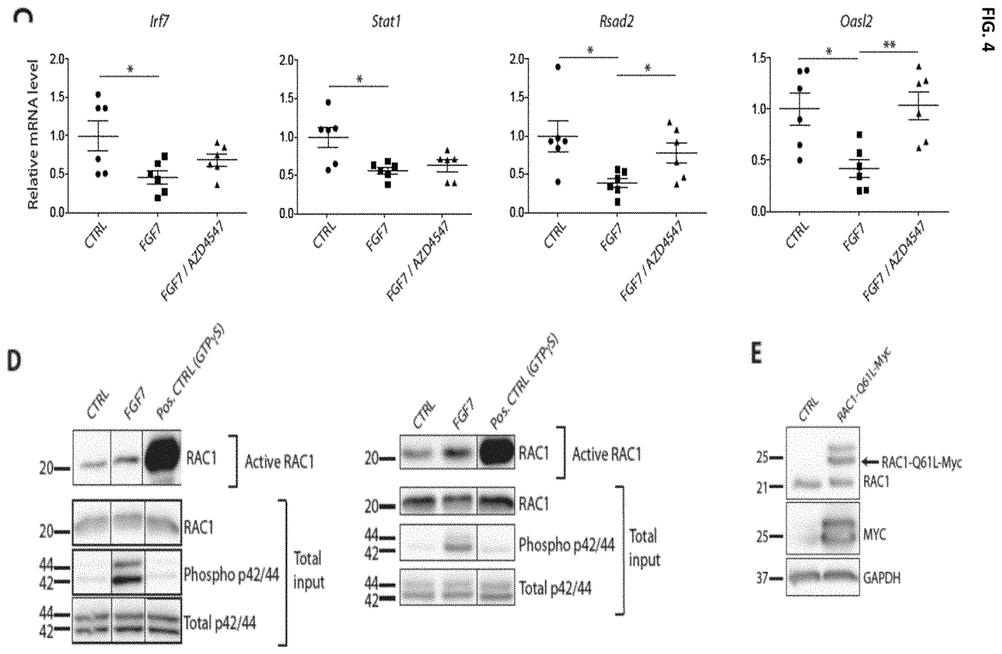

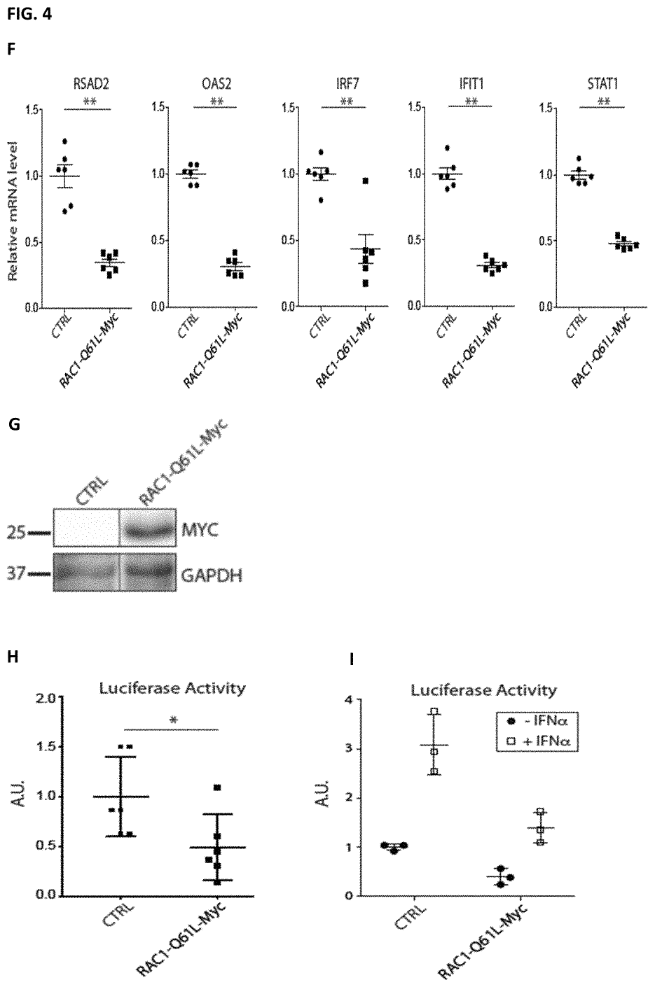

In a search for the signaling pathways that mediate the suppression of ISG expression by FGF7, it was shown that inhibition of p38 MAP kinase had a major effect (FIG. 4A). In addition, the RAC1 inhibitor NSC23766 partially or even completely rescued the FGF7-mediated ISG downregulation (FIG. 4B). The same effect was observed with AZD4547, a selective inhibitor of the FGFR1, FGFR2 and FGFR3 kinase activities (Gavine, Cancer Res 72, 2045-2056, 2012) (FIG. 4C). Consistent with a role of RAC1 in the effect of FGF7 on ISG expression, FGF7 activated RAC1 in HaCaT cells, and expression of a constitutively active RAC1 mutant (Q61L mutation) in HaCaT cells suppressed ISG expression (FIG. 4D-F). This mutant also suppressed ISRE-mediated transcription of the luciferase reporter gene in HEK293 cells (FIG. 4G, H), even in the presence of IFNα (FIG. 41).

### Example 7—FGF Signaling Promotes Replication of Herpes Simplex Virus in Keratinocytes

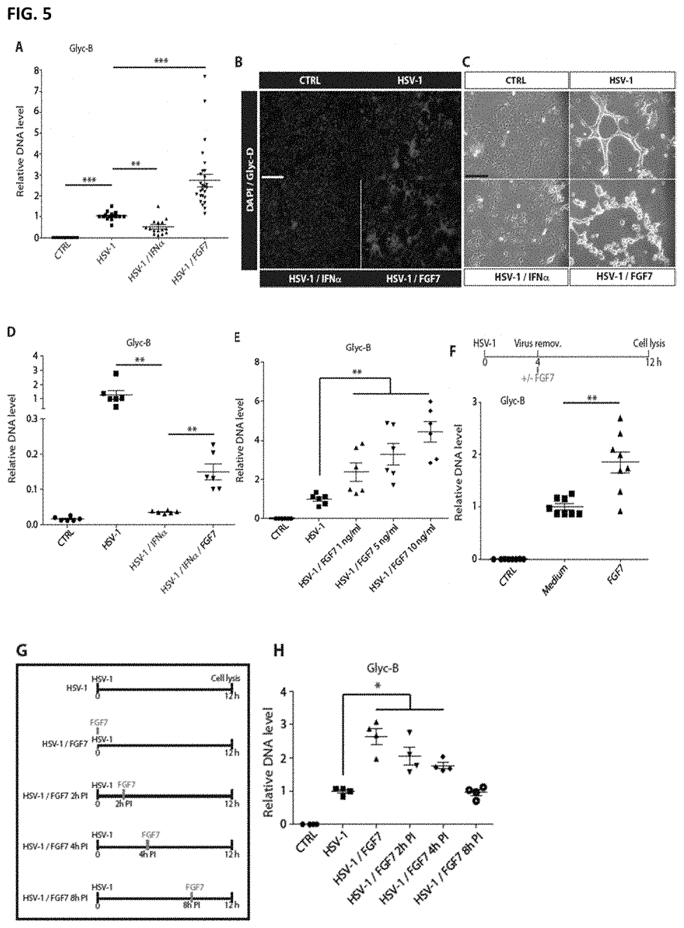

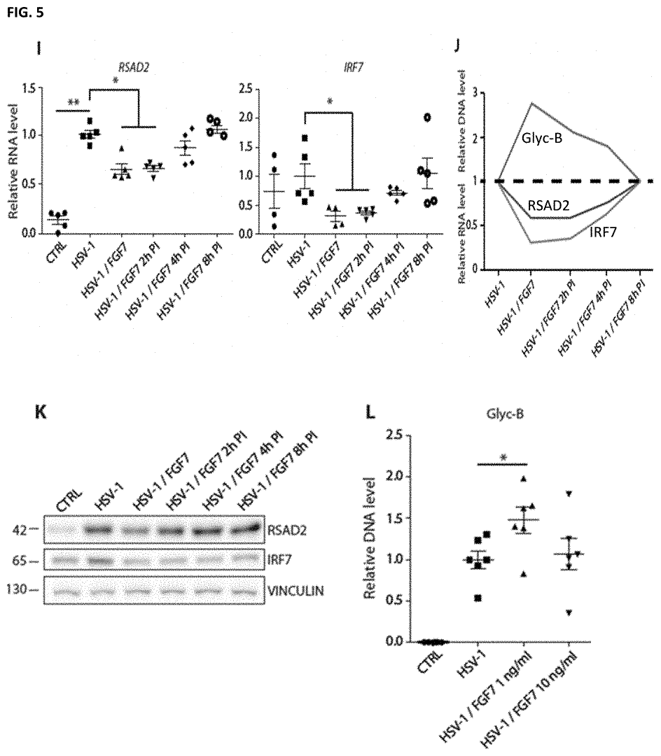

Due to the strong anti-viral activity of the products of ISGs, it was tested if modulation of FGF signaling affects viral infection and/or replication. Since human keratinocytes are the first entry sites for Herpes Simplex Virus type 1 (HSV-1) (Petermann et al., J. Virol 89, 262-274, 2015), HSV-1 was used for this purpose. Sub-confluent HaCaT cells were infected with HSV-1 (MOI=0.5) in the presence or absence of FGF7 and/or IFNα. 16h later, the amount of viral glycoprotein B (Glyc-B) DNA was determined in the infected cells. A strong increase in viral DNA was seen upon FGF7 treatment, while IFNα had the opposite effect (FIG. 5A). This was confirmed by immunofluorescence staining of the infected cells for the HSV-1 glycoprotein D (Glyc-D), which increased strongly in response to FGF7 (FIG. 5B). Within 48 h after infection, HSV-1 infected cells had fused, but they still attached to the dish. In the presence of FGF7, however, they were completely detached (FIG. 5C). IFNα-treated HSV-1 infected cells were almost indistinguishable from non-infected cells (FIG. 5C). Importantly, the IFNα-mediated reduction in viral DNA was partially reverted by FGF7 (FIG. 5D). FGF7 induced the levels of viral DNA in a dose-dependent manner (FIG. 5E). Thus, FGF7 is a potent inducer of HSV-1 infection/replication in keratinocytes.

To determine if FGF7 influences viral infection and/or replication, HaCaT cells were infected with HSV-1 for 4 hours. The virus was then removed and cells were treated with FGF7 for 8 hours before harvesting. A strong increase in viral DNA upon FGF7 treatment (FIG. 5F) was still observed under these conditions, indicating that FGF7 promotes viral replication. To determine if this effect is exerted via suppression of ISG expression, HSV-1 infected HaCaT cells were treated with FGF7 for different time periods (FIG. 5G). FGF7 strongly promoted HSV-1 replication when it was added together with the virus, or 2h or 4h post infection. Thus, cells were exposed to FGF7 for 12h, 10h or 8h in these experiments. However, when FGF7 was only present for 4h, viral replication was no longer promoted (FIG. 5H). Importantly, the extent of viral protein production (Glyc-B) negatively correlated with ISG expression at the mRNA and protein levels (FIG. 5H-K). This result suggests that FGF7 mainly affects viral replication by regulating ISG expression.

The effect of FGF7 on HSV-1 infection was confirmed with human primary keratinocytes (FIG. 5L).

### Example 8—FGF Signaling Promotes Replication of Different Viruses

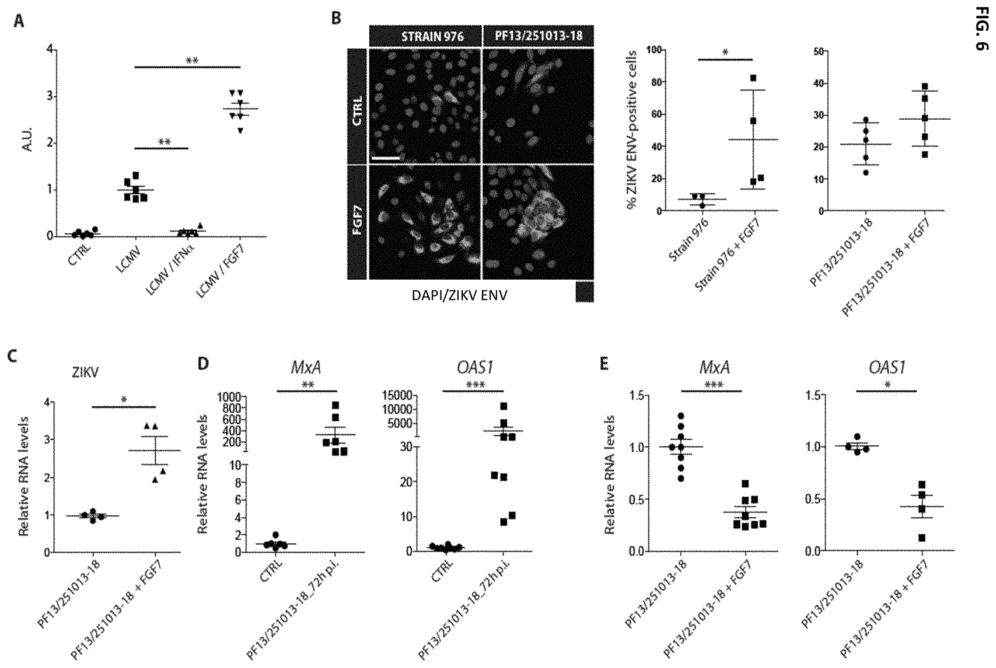

The effect of FGF7 on viral replication is not restricted to HSV-1. This was revealed by infection studies with Lymphocytic Choriomeningitis Virus (LCMV), a murine pathogen, which can also infect human cells (Welsh et al., Curr Protocol Microbiol, Chapter 15, 2008). When HaCaT cells were infected with LCMV and immediately exposed to FGF7, expression levels of the LCMV nucleoprotein (NP) were strongly increased in the presence of FGF7, while IFN-α blocked the viral replication (FIG. 6A).

Next, HaCaT cells were infected with two different strains of Zika Virus (ZIKV), a major human pathogen. FGF7 treatment increased the number of cells expressing the ZIKV envelope protein (FIG. 6B). Analysis of ZIKV gene expression revealed that FGF7 was also efficient when added 2 h after the infection (FIG. 6C), thus confirming that FGF7 influences viral replication. Consistent with the important role of ISGs in the effect of FGF7, expression of MxA and OAS1, two highly expressed ISGs during infection by viruses belonging to the Flaviviridae family, strongly increased during ZIKV infection (FIG. 6D), and this increase was strongly suppressed in the presence of FGF7 (FIG. 6E).

### Example 9—Inhibition of the FGFR-RAC1 Pathway Inhibits Viral Replication In Vitro, Ex Vivo and In Vivo

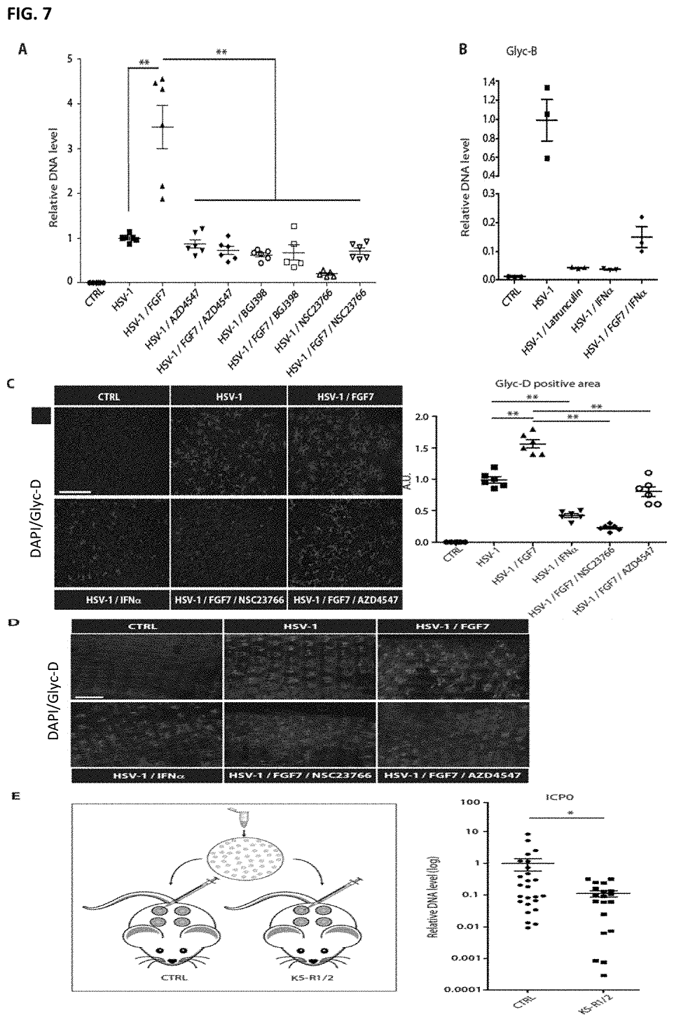

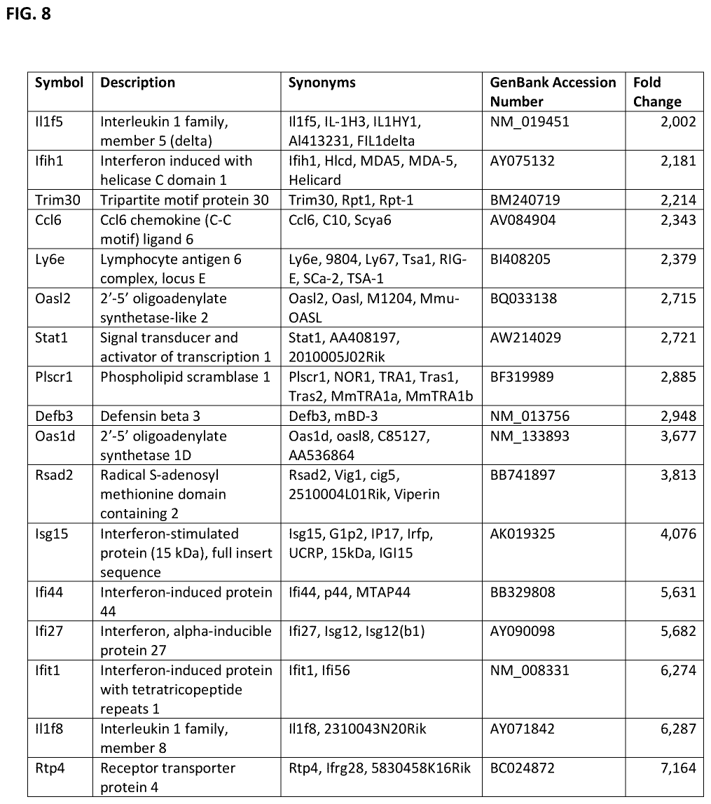

To determine the importance of the FGFR/RAC1 signaling pathway for the viral life cycle, HSV-1 infected HaCaT cells were treated with FGF7 in the presence or absence of the RAC1 inhibitor NSC23766 or the FGFR kinase inhibitors AZD4547 or BGJ398 (Guagnano et al., J. Med. Chem, 7066-7083, 2011). After 24 h, Glyc-B DNA levels were strongly reduced in NSC23766 treated cells, even in the presence of FGF7. Both FGFR kinase inhibitors also inhibited the positive effect of FGF7 on viral replication (FIG. 7A). These results were verified by immunofluorescence staining for Glyc-D. NSC23766 or AZD4547 treatment strongly reduced the amount of Glyc-D-positive fused cells similar to IFNα treatment and also their size (FIG. 7C).

Next, the effect of FGF7 on HSV-1 replication in epidermal sheets from the tails of wildtype mice was determined ex vivo by incubating them with HSV-1 alone or in combination with FGF7 and with RAC1 or FGFR kinase inhibitors. After 48 h, the sheets were stained with the antibody against Glyc-D to monitor the infection. HSV-1 preferentially infected cells of the hair follicles in the absence of FGF7. The staining of the hair follicles was stronger in the presence of FGF7, and under these conditions virus-infected cells were present throughout the interfollicular epidermis (FIG. 7D). Viral dissemination was potently suppressed by RAC1 or FGFR kinase inhibition (FIG. 7D), consistent with the in vitro data.

Finally, the effect of FGFR deficiency on HSV-1 replication in vivo was examined using K5-R1/R2 mice. 48 h after subcutaneous inoculation of HSV-1, the amount of viral immediate-early protein ICP0 DNA was determined in the infected epidermis. There was a significant reduction in viral replication in K5-R1/R2 compared to control mice (FIG. 7E). Taken together, these results demonstrate that inhibition of FGFR signaling inhibits viral replication in vitro, ex vivo and in vivo.

1.-15. (canceled)

16. A method for the therapeutic or prophylactic treatment of a viral disease, comprising the steps of:
(a) providing a subject in need thereof; and
(b) administering a pharmaceutically effective amount of at least one inhibitor of FGFR kinase activity, wherein the method is effective in the therapeutic or prophylactic treatment of the viral disease.

17. The method of claim 16, wherein the viral disease is caused by a virus selected from the group consisting of Herpes simplex virus (HSV) 1 and/or 2, Human Papilloma viruses, Ebola virus, Marburg virus, Venezuelan Equine Encephalitis virus, Chikungunya virus, Easter Equine Encephalitis virus, Western Equine Encephalitis virus, Monkey Pox virus, Corona virus, Respiratory Syncytial virus, Adenovirus, Human Rhinovirus, Influenza virus, HIV, HCV, Norovirus, Saporovirus, Cytomegalovirus (CMV), Dengue virus, West Nile virus, Yellow fever virus, Zika Virus, Lymphocytic Choriomeningitis virus (LCMV), and HBV,

18. The method of claim 16, wherein the subject in need is selected from the group consisting of mammal, human, and bird.

19. The method of claim 16, wherein administration of the at least one inhibitor of FGFR kinase activity is by at least one of oral, intranasal, intravenous, intramuscular, intradermal, subcutaneous, intraperitoneal or topical administration.

20. The method of claim 16, wherein the at least one inhibitor of FGFR kinase activity comprises one or more of:
(i) FGFR kinase inhibitors: AZD4547, Ponatinib, Dovitinib, Nintedanib, Lenvatinib, Lucitanib, Brivanib, ENMD-2076, BGJ398, FGF401, Lucitanib, PD173074, SU5402, SSR128129E, ARQ 087, LY2874455, Debio 1347, TAS-120, Erdafitinib, Nintedanib, Orantinib;
(ii) FGFR ligand trap: FP1039;
(iii) FGFR neutralizing antibodies: IMC-A1, PRO-001, R3Mab, FPA144, MGFR1877S; or
(iv) physiologically acceptable salts thereof.

21. The method of claim 16, wherein the at least one inhibitor of FGFR kinase activity comprises one or more of AZD4547, BGJ398, LY2874455, Debio 1347, TAS-120, Erdafitinib, FPA144, FP1039, or physiologically acceptable salts thereof.

22. The method of claim 16, wherein:
(a) the at least one inhibitor of FGFR kinase activity comprises one or more of AZD4547, BGJ398, FP1039, FPA144, or physiologically acceptable salts thereof; and
(b) the viral infection is caused by a virus selected from the group consisting of Herpes simplex virus (HSV) 1 and/or 2, Human Papilloma viruses, Ebola virus, Marburg virus, Venezuelan Equine Encephalitis virus, Chikungunya virus, Easter Equine Encephalitis virus, Western Equine Encephalitis virus, Monkey Pox virus, Corona virus, Respiratory Syncytial virus, Adenovirus, Human Rhinovirus, Influenza virus, HIV, HCV, Norovirus, Saporovirus, Cytomegalovirus (CMV), Dengue virus, West Nile virus, Yellow fever virus, Zika Virus, Lymphocytic Choriomeningitis virus (LCMV), and HBV.

23. The method of claim 16, wherein:
(a) the at least one inhibitor of FGFR kinase activity comprises one or more of:
(i) FGFR kinase inhibitors: AZD4547, Ponatinib, Dovitinib, Nintedanib, Lenvatinib, Lucitanib, Brivanib, ENMD-2076, BGJ398, FGF401, Lucitanib, PD173074, SU5402, SSR128129E, ARQ 087, LY2874455, Debio 1347, TAS-120, Erdafitinib, Nintedanib, Orantinib;
(ii) FGFR ligand traps: FP1039;
(iii) FGFR neutralizing antibodies: IMC-A1, PRO-001, R3Mab, FPA144, MGFR1877S; or
(iv) physiologically acceptable salts thereof; and
(b) the viral infection is caused by a virus selected from the group consisting of Dengue virus, HSV1, HSV2, HIV1, HIV2, HCV, Zika Virus, Lymphocytic Choriomeningitis virus (LCMV), Influenza virus, SARS virus, and MARS virus.

24. The method of claim 16, wherein:
(a) the at least one inhibitor of FGFR kinase activity comprises one or more of AZD4547, BGJ398, FP144 or physiologically acceptable salts thereof; and
(b) the viral infection is caused by a virus selected from the group consisting of HSV1, HSV2, Lymphocytic Choriomeningitis virus (LCMV), and Zika Virus.

25. The method of claim 16, wherein:
(a) the at least one inhibitor of FGFR kinase activity inhibits the kinase activity of at least one of FGFR1, FGFR2, FGFR3 or a combination thereof; and
(b) the viral infection is caused by a virus selected from the group consisting of viruses located in epithelial cells of the skin (keratinocytes), an HSV1 infection in keratinocytes, and an HSV2 infection in keratinocytes.

26. The method of claim 16, wherein:
(a) the at least one inhibitor of FGFR kinase activity comprises an FGFR ligand trap binding ligands of FGFR2b; and
(b) the viral infection is caused by a virus selected from the group consisting of viruses affecting epithelial cells of the skin (keratinocytes), an HSV1 infection in keratinocytes, and an HSV2 infection in keratinocytes, viruses affecting the lung, and an influenza virus infection of the lung.

27. The method of claim 16, wherein:
(a) the at least one inhibitor of FGFR kinase activity inhibits the kinase activity of at least one of FGFR1, FGFR2, FGFR3, FGFR4 or a combination thereof; and
(b) the viral infection is caused by a virus selected from the group consisting of viruses affecting T cells, and an HIV infection of T cells.

28. The method of claim 16, wherein:
(a) the at least one inhibitor of FGFR kinase activity inhibits the kinase activity of at least one of FGFR1, FGFR2, FGFR3, FGFR4 or a combination thereof; and
(b) the viral infection is caused by a virus selected from the group consisting of viruses affecting hepatocytes, an HCV infection, and an HBV infection.

29. The method of claim 16, wherein:
(a) the at least one inhibitor of FGFR kinase activity comprises one or more of FP1039, FPA144, AZD4547, or BGJ398; and
(b) the viral infection is caused by a virus selected from the group consisting of viruses affecting keratinocytes, an HSV1 infection in keratinocytes, and an HSV2 infection in keratinocytes.

30. The method of claim 16, wherein the at least one inhibitor of FGFR kinase activity is administered with one or more physiologically acceptable excipients.

31. The method of claim 16, wherein the at least one inhibitor of FGFR kinase activity is formulated for topical, oral, intravenous, intranasal, or rectal administration.

32. The method of claim 16, wherein:
(a) the at least one inhibitor of FGFR kinase activity comprises one or more of AZD4547, BGJ398, FPA144 or physiologically acceptable salts thereof;
(b) the at least one inhibitor of FGFR kinase activity is administered by at least one of oral, intranasal, intravenous, intramuscular, intradermal, subcutaneous, intraperitoneal, or topical administration; and
(c) the viral infection is an HSV-1 infection.

33. The method of claim 32, wherein the viral infection is an HSV 1 infection of keratinocytes and the at least one inhibitor of FGFR kinase activity is administered topically.
